SEQUENCE LISTING (19 sequences)

SEQ ID NO: 1 - DNA, 1999 bp, Homo sapiens
  source 1..1999: mol_type = other DNA
taatacgactcactataaggagaaagcttacatttgcttctgacacaactgtgttcactagcaacctcaaacagacaccatggccttaccagtgaccgccttgctcctgccgctggccttgctgctccacgccgccaggccggctagccaggtgcagctggtgcagagcggcgcggaagtgaaaaaaccgggcagcagcgtgaaagtgagctgcaaagcgagcggctatacctttaccagctatgtgatgcattgggtgcgccaggcgccgggccagggcctggaatggatgggctatattattccgtataacgatgcgaccaaatataacgaaaaatttaaaggccgcgtgaccattaccgcggataaaagcaccagcaccgcgtatatggaactgagcagcctgcgcagcgaagataccgcggtgtattattgcgcgcgctataactatgatggctattttgatgtgtggggccagggcaccctggtgaccgtgagcagcggcggcggcggcagcggcggcggcggcagcggcggcggcggcagcgatgtggtgatgacccagagcccggcgtttctgagcgtgaccccgggcgaaaaagtgaccattacctgccgcgcgagccagagcattagcgattatctgcattggtatcagcagaaaccggatcaggcgccgaaactgctgattaaatatgcgagccagagcattagcggcgtgccgagccgctttagcggcagcggcagcggcaccgattttacctttaccattagcagcctggaagcggaagatgcggcgacctattattgccagaacggccatagctttccgccgacctttggcggcggcaccaaagtggaaattaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 2 - DNA, 19 bp, Homo sapiens
  source 1..19: mol_type = other DNA
taatacgactcactataag

SEQ ID NO: 3 - DNA, 60 bp, Homo sapiens
  source 1..60: mol_type = other DNA
gagaaagcttacatttgcttctgacacaactgtgttcactagcaacctcaaacagacacc

SEQ ID NO: 4 - DNA, 1570 bp, Homo sapiens
  source 1..1570: mol_type = other DNA
taatacgactcactataaggagaaagcttacatttgcttctgacacaactgtgttcactagcaacctcaaacagacaccatggccttaccagtgaccgccttgctcctgccgctggccttgctgctccacgccgccaggccggctagccaggtgcagctggtgcagagcggcgcggaagtgaaaaaaccgggcagcagcgtgaaagtgagctgcaaagcgagcggctatacctttaccagctatgtgatgcattgggtgcgccaggcgccgggccagggcctggaatggatgggctatattattccgtataacgatgcgaccaaatataacgaaaaatttaaaggccgcgtgaccattaccgcggataaaagcaccagcaccgcgtatatggaactgagcagcctgcgcagcgaagataccgcggtgtattattgcgcgcgctataactatgatggctattttgatgtgtggggccagggcaccctggtgaccgtgagcagcggcggcggcggcagcggcggcggcggcagcggcggcggcggcagcgatgtggtgatgacccagagcccggcgtttctgagcgtgaccccgggcgaaaaagtgaccattacctgccgcgcgagccagagcattagcgattatctgcattggtatcagcagaaaccggatcaggcgccgaaactgctgattaaatatgcgagccagagcattagcggcgtgccgagccgctttagcggcagcggcagcggcaccgattttacctttaccattagcagcctggaagcggaagatgcggcgacctattattgccagaacggccatagctttccgccgacctttggcggcggcaccaaagtggaaattaaactcgagaagcccaccacgacgccagcgccgcgaccaccaacaccggcgcccaccatcgcgtcgcagcccctgtccctgcgcccagaggcgagccggccagcggcggggggcgcagtgcacacgagggggctggacttcgccagtgataagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 5 - DNA, 277 bp, synthetic construct
  source 1..277: mol_type = other DNA
gctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctag

SEQ ID NO: 6 - DNA, 152 bp, synthetic construct
  source 1..152: mol_type = other DNA
aaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 7 - protein, 496 aa, Homo sapiens
MALPVTALLLPLALLLHAARPASQVQLVQSGAEVKKPGSSVKVSCKASGYTFTSYVMHWVRQAPGQGLEWMGYIIPYNDATKYNEKFKGRVTITADKSTSTAYMELSSLRSEDTAVYYCARYNYDGYFDVWGQGTLVTVSSGGGGSGGGGSGGGGSDVVMTQSPAFLSVTPGEKVTITCRASQSISDYLHWYQQKPDQAPKLLIKYASQSISGVPSRFSGSGSGTDFTFTISSLEAEDAATYYCQNGHSFPPTFGGGTKVEIKLEKPTTTPAPRPPTPAPTIASQPLSLRPEASRPAAGGAVHTRGLDFASDKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - DNA, 4934 bp, Homo sapiens
  source 1..4934: mol_type = other DNA
taatacgactcactataaggagaaagcttacatttgcttctgacacaactgtgttcactagcaacctcaaacagacaccatggcgctcccagtgactgcccttctgttgccattggccttgcttttgcacgccgcgaggcccgcctcccaagtgcaactcgttcagtccggggcggaggtcaaaaagccaggctcctccgtcaaggtatcctgtaaggcgtccggatacacattcacctcctacgtcatgcactgggtcagacaagcaccgggccaagggctcgagtggatgggctacattataccgtacaacgacgcgacgaagtacaacgaaaagttcaaagggagagtaaccataacggccgacaaaagcacaagcactgcgtacatggaactctcctccctccgatccgaagataccgcagtatactactgtgccagatacaactacgatggttacttcgacgtctggggacagggcaccctggtaacagtatcatcaggaggagggggcagcggaggcggcggatcagggggcggcggcagcgacgtcgtgatgacgcagagcccggcattcctctctgtcacacccggagagaaggtcacaatcacttgccgcgcatcccaaagcatatcagactacctgcactggtaccagcagaaacccgaccaagcccccaaactcttgataaagtacgccagtcaaagcatatcaggagtcccctcccggttcagtggcagtggctccggaacggacttcacgttcaccatctcatcattggaagcggaagacgcggcaacatactactgccaaaatggccacagcttcccgcccacgttcgggggcggaacaaaagtcgaaataaagttggagaaacccaccactacaccagcccccagaccacccactcccgcaccgaccatcgcgagccagccactctctctgagacccgaggcctcacgcccggccgcagggggcgcggtccacacgcgcgggctcgattttgcctccgacaaacccttctgggtcctggtcgtagtaggaggagtcctggcctgctactccttgttggtaaccgttgcgttcatcatcttctgggtcaagagaggccgaaagaaactgctctacatcttcaagcaacccttcatgcgcccggtccaaacaacacaagaagaggacggctgctcatgccgctttccggaggaggaggaagggggctgtgaattgagggtgaaattcagccggtctgcggacgcccccgcctaccaacagggccagaatcaactctacaacgaactcaacttggggagacgcgaggaatacgatgtactggataagcgacgcgggcgcgaccctgagatggggggcaagccccagaggaggaagaacccccaagagggcctgtacaacgagctgcagaaggacaaaatggcggaggcctactcagagatcgggatgaagggcgaacggagacgcggaaaagggcacgacgggctctaccaaggcttgtcaacagctaccaaggacacctatgacgcgctccacatgcaagcgttgccacccagataagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 9 - DNA, 1975 bp, Homo sapiens
  source 1..1975: mol_type = other DNA
taatacgactcactataaggagaaagcttacatttgcttctgacacaactgtgttcactagcaacctcaaacagacaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggtggcggtggcagcggcggtggtggttccggaggcggcggttctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgatagtaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 10 - protein, 486 aa, Homo sapiens
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 4937 bp, Homo sapiens
  source 1..4937: mol_type = other DNA
taatacgactcactataaggagaaagcttacatttgcttctgacacaactgtgttcactagcaacctcaaacagacaccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 12 - protein, 497 aa, Homo sapiens
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAADYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 4958 bp, Homo sapiens
  source 1..4958: mol_type = other DNA
taatacgactcactataaggagaaagcttacatttgcttctgacacaactgtgttcactagcaacctcaaacagacaccatgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaaaaaacccggatccgtgggcgaaaaacctgaacgaaaaagattatattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcagtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaagatctggcgcgccgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacg

SEQ ID NO: 14 - protein, 504 aa, Homo sapiens
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAKNPDPWAKNLNEKDYIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1971 bp, Homo sapiens
  source 1..1971: mol_type = other DNA
taatacgactcactataaggagaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagaagcttgagaataaactagtattcttctggtccccacagactcagagagaacccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtgataagactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcgtcgactctagaaaaaaaaaaaaaaaaaaaaaaaaaaaaaaaaaaaaaaaaaggatccccgggcgagctcccaaaaaaaaaaaaaaaaaaaaaaaaaaaaaaccgaattcctgcagctcgagaaaaaaaaaaaaaaaaaaaaaaaaaaaaaaaaaaaaaaaaa

SEQ ID NO: 16 - DNA, 766 bp, synthetic construct
  source 1..766: mol_type = other DNA
gagaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagaagcttgagaataaactagtattcttctggtccccacagactcagagagaacccgccacc

SEQ ID NO: 17 - DNA, 726 bp, Homo sapiens
  source 1..726: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagtgataa

SEQ ID NO: 18 - protein, 239 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 19 - DNA, 308 bp, synthetic construct
  source 1..308: mol_type = other DNA
gactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcgtcgactctag

(19) United States
(12) Patent Application Publication
(10) Pub. No.: US 2025/0188415 A1
(43) Pub. Date: Jun. 12, 2025

(54) NK CELLS TRANSFECTED WITH CAR RNA-LNP

(71) Applicants: ProMab Biotechnologies, Inc., Richmond, CA (US); Forevertek Biotechnology Co., Ltd, Changsha (CN)

(72) Inventors: Lijun Wu, Berkeley, CA (US); Vita Golubovskaya, Pinole, CA (US)

(21) Appl. No.: 19/047,481

(22) Filed: Feb. 6, 2025

Related U.S. Application Data
(63) Continuation of application No. PCT/US2023/071898, filed on Aug. 9, 2023.
(60) Provisional application No. 63/371,025, filed on Aug. 10, 2022.

(51) Int. Cl. C12N 5/0783; A61K 35/17; A61K 40/15; A61K 40/31; A61K 40/42; C07K 16/28; C12N 15/88

(52) U.S. Cl. CPC C12N 5/0646; A61K 35/17; A61K 40/15; A61K 40/31; A61K 40/4211; C07K 16/2896; C12N 15/88; A61K 2239/13; A61K 2239/21; C07K 2317/622; C12N 2510/00

(57) ABSTRACT

The present invention provides CAR mRNA embedded to lipid nanoparticles to transfect immune NK cells and generate functional CAR-NK cells. The CAR-NK cells target tumor antigens to kill tumors. The present invention provides several advantages: transient expression, less toxicity and lower manufacturing cost for CAR-NK cells.

### CROSS-REFERENCE TO RELATED APPLICATION(S)

This application is a continuation of PCT/US2023/071898, filed Aug. 9, 2023; which claims the benefit of U.S. Provisional Application No. 63/371,025, filed Aug. 10, 2022. The contents of the above-identified applications are incorporated herein by reference in their entirety.

### REFERENCE TO SEQUENCE LISTING, TABLE OR COMPUTER PROGRAM

This application contains an ST.26 compliant Sequence Listing, which was submitted in xml format via Patent Center and is hereby incorporated by reference in its entirety. The .xml copy, created on Aug. 8, 2023, is named SequenceListing.xml and is 43100 kilobytes in size.

### FIELD OF THE INVENTION

The present application uses CAR mRNA-LNP (lipid nanoparticle) technology to effectively transfect expanded NK cells and to generate functional CAR-NK cells. The functional CAR-NK cells are effective to attack tumor cells overexpressing tumor extracellular antigen.

### BACKGROUND OF THE INVENTION

Natural Killer (NK) Cells are lymphocytes in the same family as T and B cells, coming from a common progenitor. However, as cells of the innate immune system, NK cells are classified as group I Innate Lymphocytes and respond quickly to a wide variety of pathological challenges. NK cells are best known for killing virally infected cells, and detecting and controlling early signs of cancer.

NK cells were first noticed for their ability to kill tumor cells without any priming or prior activation. They are named for this natural killing. Additionally, NK cells secrete cytokines such as IFN-γ and TNF-α, which act on other immune cells like macrophage and dendritic cells to enhance the immune response.

Immunotherapy is emerging as a highly promising approach for the treatment of cancer. NK cells as the armed forces of our immune system, constantly look for foreign antigens and discriminate abnormal (cancer or infected cells) from normal cells.

Chimeric antigen receptor (CAR)-T cells recently were approved by FDA to treat hematological cancers (leukemia, lymphoma, and multiple myeloma) and demonstrated highly promising results (1-4). CAR-T cell therapy made impressive advancement in the field of cancer therapy but has several limitations such as cytokine release storm (CRS), neurotoxicity and challenges to target solid tumors (5, 6).

Another type of promising cell therapy against cancer is CAR-NK cells (5), (7). One of the advantages of NK cells is low risk of graft-versus-host disease (GVHD) and low toxicity (8, 9). NK cells are also good candidates for allogeneic cell therapy as they are independent of HLA-TCR recognition signaling of T cells (10).

CAR-NK cells were used in preclinical studies against B-cell malignancies (7, 11, 12), multiple myeloma (13, 14), and against solid tumors such as glioblastoma (15, 16), breast (17, 18) and ovarian cancers (19). There are several clinical trials ongoing with CAR-NK cells against hematological and solid tumors (5) which support use of CAR-NK cells against different cancers.

The use of viral vectors is associated with high cost, regulatory requirements, and some safety concern (20). There are several non-viral methods such as RNA electroporation, DNA transfection, but these methods have limitations due to low efficiency of transfection (20). Development of non-viral delivery of CAR to NK cells has advantages for manufacturing due to lower cost, easier than viral CAR preparation and convenience for development of allogeneic off-the-shelf CAR-NK cells.

There exists a need to have a better delivery system for CAR.

### BRIEF DESCRIPTION OF THE DRAWINGS

FIG. 1. Cap 0 and Cap 1 structure.

FIG. 2. The scheme of DNA vector template (A) used for in vitro transcription of CAR RNA (B). 5′UTR, 5′ untranslated region; 3′UTR, 3′untranslated region; poly A tail for increased stability.

FIG. 3. The expansion of NK cells with K562-4-BB, IL-21 K562 cell line. FIG. 3 shows an average of fold of expansion of NK cell expansion from two different donors is shown.

FIG. 4. Detection of BCMA-CAR in NK cells transfected with BCMA-CAR-LNP with fluorescent by FACS with anti-mouse FAB antibody. RNA was in vitro transcribed with pseudo-UTP, with cap1. Primary Ab was from Jackson Immunoresearch (Cat #115-066-072) Biotin-SP-conjugated AffiniPure F(ab′)2 Fragment Goat Anti-Mouse IgG, F(ab′)2 fragment specific (1:100); Secondary Ab was from Biolegend, PE-Streptavidin (1:100); Live/Dead Cell were detected with 7-AAD Viability Staining Solution (1:50) from Biolegend.

FIGS. 5A-5D. BCMA-CAR-NK cells kill multiple myeloma cell lines and secrete higher levels of IFN-gamma than NK cells in a dose-dependent manner. A. RTCA killing assay with hBCMA-CAR-NK and multiple myeloma RPMI-8226 cells. B. Killing RTCA assay with hBCMA-CAR-NK and MM1S multiple myeloma cells. C. hBCMA-CAR-NK cells secrete IFN-gamma by ELISA assay in a dose-dependent manner with multiple myeloma RPM-18226 target cells (C) and MM1S target cells (D). Bars show an average of 3 independent measurements +/−standard deviations. P<0.05, Student's t-test BCMA-CAR-NK cells vs NK cells, IFN-gamma secretion vs NK cells.

FIG. 6. CD19-CAR-NK cells kill leukemia cell lines and secrete higher levels of IFN-gamma than NK cells in a dose-dependent manner. A. RTCA killing assay with CD19-CAR-NK effector cells and Daudi target cells. Bars show average cytotoxicity of effector cells in RTCA assay (Materials and Methods) from 3 independent measurements. *p<0.05 CAR-NK vs NK, Student's t-test. B. CD19-CAR-NK secrete higher levels of IFN-gamma in a dose-dependent manner than NK cells p<0.05, CD19-CAR-NK vs NK cells in secretion of IFN-gamma by ELISA. C. Cytotoxicity assay with Nallm-6-luciferase positive cells at different E:T ratios by luciferase assay (Materials and Methods). CD19-CAR-NK cell cytotoxicity was significantly higher than NK cells. *p<0.05 CAR-NK vs NK cells by Student's t-test. D. ELISA assay shows dose-dependent secretion of IFN-gamma by CD19-CAR-NK and NK cells and Nalm-6 target cells. CD19-CAR-NK cells secreted significantly higher levels of IFN-gamma than with NK cells against Nalm-6 target cells. *p<0.05, CD19-CAR-NK vs NK cells, IFN-gamma by ELISA.

FIGS. 7A-7B. CD19-CAR-NK decreased Nalm-6-luciferase tumor growth significantly more than NK cells. 7A. 1×105 Nalm-6-luciferase positive cells were injected intravenously into NSG mice. Then 5×106 frozen/thawed CD19-CAR-NK cells were injected at days 1, 3, 6, and 8 intravenously. Imaging was performed with Xenogen Ivis system. 7B. Quantification of imaging is shown. *p<0.00002, CD19-CAR-NK vs NK cells by Student's t-test.

### DETAILED DESCRIPTION OF THE INVENTION

### Definitions

As used herein, “about” refers to +10% of the recited value.

As used herein, “activated NK cells” means NK cells activated for proliferation and cell killing.

As used herein, a “chimeric antigen receptor (CAR)” is a receptor protein that has been engineered to give T cells the new ability to target a specific protein. The receptor is chimeric because they combine both antigen-binding and T-cell activating functions into a single receptor. CAR is a fused protein comprising an extracellular domain capable of binding to an antigen, a transmembrane domain, and at least one intracellular domain. The “chimeric antigen receptor (CAR)” is sometimes called a “chimeric receptor”, a “T-body”, or a “chimeric immune receptor (CIR)”. The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell.

The “intracellular domain” means any oligopeptide or polypeptide known to function as a domain that transmits a signal to cause activation or inhibition of a biological process in a cell.

As used herein, a “domain” means one region in a polypeptide which is folded into a particular structure independently of other regions.

As used herein, “feeder cells” consist in a layer of cells unable to divide, which provides extracellular secretions to help another cell to proliferate.

As used herein, “humanized antibodies” are antibodies from non-human species whose non-CDR sequences have been modified to increase their similarity to antibody variants produced naturally in humans.

As used herein, a “single chain variable fragment (scFv)” means a single chain polypeptide derived from an antibody which retains the ability to bind to an antigen. An example of the ScFv includes an antibody polypeptide which is formed by a recombinant DNA technique and in which Fv regions of immunoglobulin heavy chain (H chain) and light chain (L chain) fragments are linked via a spacer sequence. Various methods for engineering an ScFv are known to a person skilled in the art.

As used herein, a “tumor antigen” means a biological molecule having expression of which causes cancer.

Natural Killer (NK) cells are type of cytotoxic lymphocytes which are critical for innate immune system. Engineering NK cells with chimeric antigen receptor (CAR) allows CAR-NK cells to target tumor antigens. The present application uses CAR mRNA-LNP (lipid nanoparticle) technology to effectively transfect expanded from primary PBMC NK cells and to generate functional CAR-NK cells. The nanoparticle-based mRNA delivery provides many advantages, such as high stability, bioavailability, solubility, and low toxicity.

In a first aspect, the present invention provides a method for expanding NK (natural killer) cells. The method comprises: obtaining mitomycin-treated or gamma ray-irradiated K562 feeder cells that express IL21, a transmembrane domain (e.g., CD8 or CD28), and 4-1BB Ligand (41BBL), combining NK cells and the treated K562 feeder cells in a proper ratio, and incubating the mixture in an expansion medium comprising IL-2. and IL-15, and expanding the NK cells.

In the present method, a transmembrane domain such as CD8 transmembrane domain is important to hold IL15 on the cell surface.

The ratio of NK cell number to the feeder cell number is about 1:2 to 1:1, or about 1:1.

In the present method, wherein the NK cells are expanded about at least 500 fold, or at least 1000 fold. For example, the NK cells are expanded 500-1000 fold, 500-1500 fold, 500-2000 fold, or 1000-3000 fold, or more than 5000 fold such as 5000-10.000 fold.

In one embodiment, the expanded NK cells are frozen for storage. In one embodiment, the expanded NK cells are frozen in a freezing medium of CS5 or D10.

The NK cells may be obtained from PBMC, cord blood, or induced pluripotent stem (iPS) cells.

In one embodiment, for example, in research laboratories, 12-well, 6 well plates, T25, T75 well flasks are used for expansion of NK cells.

In another embodiment, for example, for a larger scale and for closed system manufacturing, the NK cells are expanded in a G-rex (Gas Permeable Rapid expansion) system, in which the scale can be from 40 ml to 1 liter.

In one embodiment, NK cells are expanded using K562 feeder cells with overexpression of 41BBL and IL21.

In one embodiment, NK cells are expanded using a solid phase such as plates coated with IL-21, IL15, 41BBL or combination of them.

NK cells can also be expanded with P21 particles, which is a membrane fraction of K562-IL21, 41BBL cells, to decrease possible safety concern on using leukemia cells in clinic.

Mitomycin or irradiation are used for stopping growth of K562 cells.

In a second aspect, the present invention is directed to NK cells transfected with mRNA and lipid nanoparticles (LNPs) complex, wherein the mRNA comprises (i) 5′-UTR (untranslated region) coding sequence, (ii) a chimeric antigen receptor fusion protein (CAR) coding sequence that targets a tumor antigen, (iii) a 3′-UTR coding sequence, and (iv) a poly A tail sequence.

The CAR comprises from N-terminus to C-terminus: (i) a single-chain variable fragment (scFv) against the tumor antigen, (ii) a transmembrane domain, (iii) at least one co-stimulatory domains, and (iv) an activating domain.

In one embodiment, the tumor antigen is BCMA, Her-2, HER-2-t2A-GM-CSF, CD47, CD19, CS1, or Claudin 18.2,

The mRNAs are embedded to in LNPs with an average size in the range of 30-250 nm, or 50-150 nm, or 70-120 nm.

The CAR expressed in NK cells can be detected against scFv antibodies, using anti-mouse or anti-human FAB detecting any Scfv, or using different tag antibody to detect ScFv with fused tag (Flag, c-myc, HA, His, TF, or any other tag). The tag is useful when no antibody known to detect scFv.

We have generated different CAR RNA and delivered to activated NK cells to target tumor cells or mating tumor cells. There are several advantages of this delivery: one advantage is to lower cost in manufacturing CAR. mRNAs are generated, delivered inside LNP-nanoparticles by transfection to immune cells and then CAR RNA is translated inside immune cells. Another advantage is there is no viral delivery of CAR that can potentially cause insertion of sequences in different genomic sites to generate unfavorable effects. The third advantage is the CAR delivery by mRNA is transient compared to viral which persists for several weeks. All these advantages can generate safer CAR-NK or other types of immune cells to be used against cancer.

The present invention also provides a method for producing CAR in NK cells. The method comprises the steps of: obtaining a mRNA-LNP complex, obtaining NK cells that have been expanded at least 500 fold, transfecting the mRNA-encapsulated LNPs into the expanded NK cells, and translating the mRNA in the NK cells to produce CAR. This method can be used in clinic for manufacturing of allogenic CAR-NK cells.

In one embodiment, the lipid nanoparticles comprise 8-[(2-hydroxyethyl) [6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000). [LNP-102 (ii)]

In one embodiment, the lipid nanoparticles comprise 8-[(2-hydroxyethyl) [6-oxo-6-(undecyloxy)hexyl]amino]-octanoic acid, 1-octylnonyl ester (SM-102), distearoylphosphatidylcholine (DSPC), Cholesterol, and 1,2-distearoyl-sn-glycero-3-phosphoethanolamine-N-[maleimide (polyethylene glycol)-2000] (DSPE-PEG2000-MAL). [LNP-102 (i)]

In one embodiment, the lipid nanoparticles comprise 2-hexyl-decanoic acid, 1,1′-[[(4-hydroxybutyl)imino]di-6,1-hexanediyl] ester (ALC-0315), DSPC, Cholesterol, and α-[2-(ditetradecylamino)-2-oxoethyl]-ω-methoxy-poly(oxy-1,2-ethanediyl) (ALC-0159). [LNP-315]

Insertion of mRNA into LNP nanoparticles provides protection of mRNA from degradation and increases the stability mRNA; mRNA is then released from LNPs into cells in vivo to generate protein. mRNA-lipid nanoparticle preparation is described in Schoenmaker (International J. Pharmaceutics, 601:120856, 2021), the article is incorporated herein by reference in its entirety, in particular regarding the LNPs.

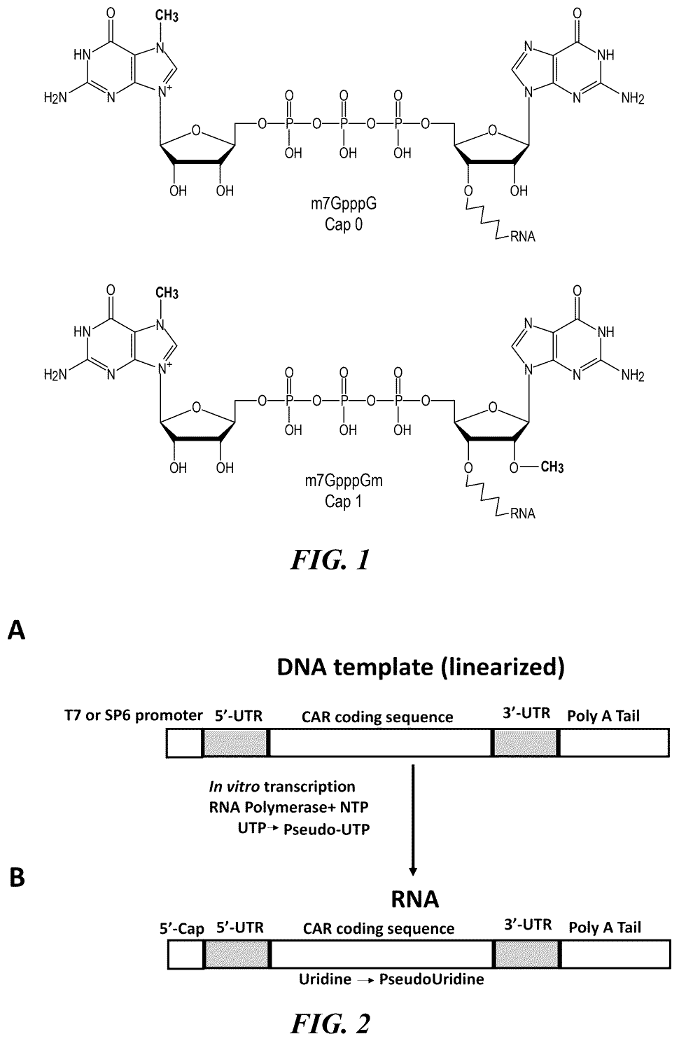

In a preferred embodiment, the mRNA further comprises 5′-cap, 5-end to (i) 5′-UTR. 5′-cap stabilizes mRNA. All eukaryotic mRNA contains a cap structure—an N7-methylated guanosine linked to the first nucleotide of the RNA via a reverse 5′ to 5′ triphosphate linkage (FIG. 1, Cap 0). In addition to its essential role of cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. It also acts as the anchor for the recruitment of initiation factors that initiate protein synthesis and the 5′ to 3′ looping of mRNA during translation. 2′O methylation of +1 nucleotide (Cap 1) may central to the non-self discrimination of innate immune response against foreign RNA. Cap 0 and Cap 1 structures are shown in FIG. 1.

The mRNA is transcribed with RNA polymerase in vitro from a DNA sequence comprising (a) a promoter coding sequence, (b) the 5′-UTR coding sequence, (c) the CAR coding sequence, (d) the 3′-UTR coding sequence, and (e) the poly A tail sequence. The poly A tail sequence improves stability and protein translation.

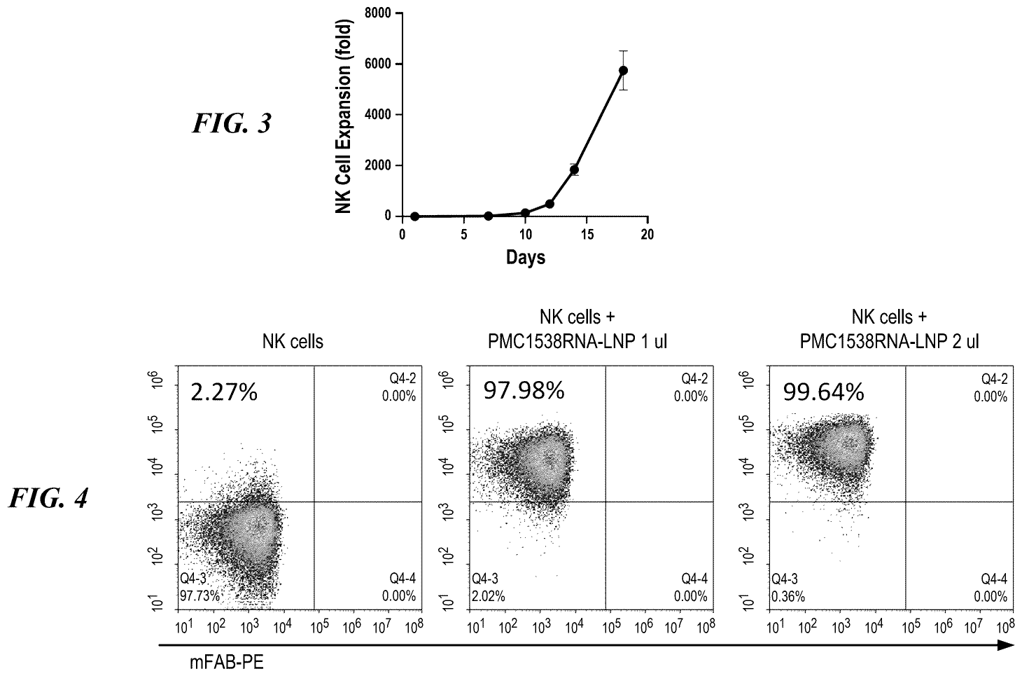

FIG. 2 shows linearized DNA template to be used for in vitro transcription with RNA polymerase and NTP to generate CAR mRNA. The DNA template contains T7 or SP6 promoter, then 5′UTR (untranslated region), the coding region of CAR, then 3′UTR and >100 poly A tail for RNA stability. The generated mRNA contains a 5′-cap such as Cap 0, Cap1, or ARCA for increased stability. The mRNAs can be either transfected to NK cells. For in vivo use, the mRNAs are added to LNPs to provide protection from degradation and to increase stability and then they are released into NK cells in vivo to generate protein.

In the DNA sequence, the promoter may be T7, T7AG promoter. Poly A tail sequence is from 20-170 nucleotides. Poly A tail sequence optionally comprises one or more linkers in between the poly A segments. If poly A tail is longer than 60 nucleotides, than it typically contains a linker which includes non-adenosine nucleotides. A linker is 5-30 or 5-25 nucleotides, e.g., 10 nucleotides or 20 nucleotides. In yet another example, poly A tails is 150-160 nucleotides in length, consisting of a two linker sequences.

DNA expression is finely regulated at the post-transcriptional level. Untranslated regions are not translated into amino acids. However, UTRs of mRNAs may control the translation, degradation and localization of stem-loop structures, upstream initiation codons and open reading frames, internal ribosome entry sites and various cis-acting elements that are bound by RNA-binding proteins. UTRs are important in the post-transcriptional regulation of DNA expression, including modulation of the transport of mRNAs out of the nucleus and of translation efficiency, subcellular localization, and stability.

5′-UTR typically has 10-1000 nucleotides, or 20-500 nucleotides, or 30-200 nucleotides, or 30-100 nucleotides. For example, 5′-UTR is 40-60 nucleotides (e.g., 50 nucleotides). 3′-UTR typically has 10-3000 nucleotides, for example, 50-500 nucleotides, or 100-300 nucleotides. Preferred 5′-UTRs and 3′-UTRs are UTRs of β-globin, or UTRs of Pfizer COVID vaccine.

β-Globin gene is shown in:

www.ncbi.nlm.nih.gov/nucleotide/V00497.1?report=genbank&log$=nuclalign&blast_rank=5&R ID=TDDZ1K98016

In one embodiment, the 5′-untranslated region is derived from human alpha-globin RNA with an optimized Kozak sequence. The 3′-untranslated region comprises two sequence elements derived from the amino-terminal enhancer of split (AES) mRNA and the mitochondrial encoded 12S ribosomal RNA to confer RNA stability and high total protein expression.

Any suitable vector, such as Vector pSP64 Poly(A) (Promega) or pGEM3Z-Vektor (Promega) can be used as a cloning vector for the DNA sequence described above.

For example, to engineer the pEM3Z-β-globin UTR-UTR-poly A tail, the 3′-UTR of the β-globin molecule flanked by restriction enzyme site can be amplified from human bone marrow. For example, a single (pEM3Z-1β-globin-UTR-A[120]) or 2 serial fragments (pEM3Z-2β-globin-UTR-A[120]) can be inserted in front of the poly(A) tail.

In general, a chimeric antigen receptor fusion protein (CAR) comprises from N-terminus to C-terminus: (i) a single-chain variable fragment (scFv) against a tumor antigen, (ii) a transmembrane domain, (iii) at least one co-stimulatory domains, and (iv) an activating domain.

In CAR, the co-stimulatory domain is selected from the group consisting of CD28, 4-1BB, GITR, ICOS-1, CD27, OX-40 and DAP10 domains. A preferred the co-stimulatory domain is CD28 or 4-1BB.

In CAR, a preferred activating domain is CD3-zeta (CD3 Z or CD3ζ).

In CAR, the transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor α or β chain, a CD3 zeta chain, CD28, CD3ε., CD45, CD4, CD5, CD8, CD9, CD16, CD22, CD33, CD37, CD64, CD80, CD86, CD134, CD137, ICOS, CD154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. Optionally, a short oligopeptide linker or a polypeptide linker, for example, a linker having a length of 2 to 10 amino acids can be arranged between the transmembrane domain and the intracellular domain. In one embodiment, a linker sequence having a glycine-serine continuous sequence can be used.

Different CARs against different tumor antigens are inserted into DNA template vector with T7 promoter for RNA polymerase to generate CAR mRNA by in vitro transcription. Tumor antigens include BCMA, Her-2, HER-2-t2A-GM-CSF, CD47, CD19, CS1, or Claudin 18.2. Then the mRNA is transfected into expanded immune cells such as primary NK cells, which is then used to kill tumor cells. In one embodiment, the NK cells are expanded with either P21 particles of K562 cells expressing 41BBL and IL21, or NK-92 cells to translate inside these cells CAR. In one embodiment, the expanded NK cells are frozen and thawed before transfected with mRNA.

Different cytokines (secreted or tethered to membrane) or ligands can be added to CAR after T2A self-cleaving peptide (EGRGSLLTCGDVEENPGP) which is added after CAR sequence before stop codon and allows cleavage of translated protein before last P of its sequence to generate two proteins CAR and added cytokine or other protein. The cytokines or ligands can be IL-12, IL15, GM-CSF, IL-2, IL-18, or FLT-3, to decrease exhaustion of NK cells and stimulate their activity.

This invention shows that freezing medium of CAR-NK cells is important to keep high viability and expression of CAR with functional killing activity and secretion of IFN-gamma against antigen-positive target cells. Donor selection is important for high activity of CAR-NK cells.

This invention shows that NK cells should be expanded at least 500-2000-fold before RNA-LNP transfection to have high killing activity and INF-gamma secretion.

The present application demonstrates non-viral delivery of CAR mRNA to expanded NK cells from primary PBMC cells using mRNA-LNP technology. NK cells were expanded from primary PBMC using K562 feeder cells expressing 4-1BB ligand and membrane-bound IL-21 which activate NK cell activity. This application demonstrates high expansion of NK cells (more than 5000-fold) and high efficiency of CAR mRNA-LNP delivery resulting in >75% CAR-positive NK cells. In addition, BCMA and CD19-CAR-NK cells effectively killed multiple myeloma and lymphoma cancer cells, respectively, and secreted high levels of IFN-gamma. In addition, CD19-CAR-NK cells significantly blocked Nalm-6 leukemia tumor growth in vivo. The present application demonstrates that CAR-NK generated with mRNA-LNP are highly functional in vitro and in vivo and are useful for future preclinical and clinical applications.

The following examples further illustrate the present invention. These examples are intended merely to be illustrative of the present invention and are not to be construed as being limiting.

### EXAMPLES

### Cells

HEK-293 cells, K562, Daudi, Nalm-6, MM1S, RPMI-8226 cell lines were purchased from ATCC, and were cultured either in RPMI-1640 or in Dulbecco's Modified Eagle's Medium (DMEM) medium with 10% FBS and penicillin/streptomycin. Nalm-6-luciferase, EGFP-positive positive cell line was obtained after transducing with luciferase-positive, EGFP positive lentivirus. K562-41BBL-IL21 (transmembrane, TM)+feeder cells were obtained after transduction of K562 cells with lentivirus containing 4-1BBL and IL21 TM coding region sequences. Human peripheral blood mononuclear cells (PBMCs) were isolated from whole blood obtained in the Stanford Hospital Blood Center, Stanford according to IRB-approved protocol (#13942). PBMC cells were isolated by standard density sedimentation over Ficoll-Paque (GE Healthcare) and cryopreserved for later use. All cell lines were cultured in a 5% CO2 incubator.

### Antibodies

Goat Anti-Mouse IgG, F(ab′)2 fragment antibodies were obtained from (Jackson Immunoresearch. Anti-Flag tag and Secondary PE-Streptavidin antibodies and 7-AAD Viability Staining Solution were obtained from Biolegend. 4-1BB ligand, IL-21 antibodies were from Biolegend. Isotype, CD3, and CD56 antibodies were from Biolegend.

### Lentivirus Generation

EGFP, Luciferase, 4-1BBL, IL21 lentiviruses were generated using HEK-293 cells as described in (24). The lentiviruses were used for transduction of different cell lines and protein expression was verified by FACS or luciferase assay.

### FACS

FACS was performed as described in (24, 28, 29). In brief, 0.25 million cells were suspended in 100 μL of buffer (PBS containing 2 mM EDTA pH 8 and 0.5% BSA) and incubated on ice with 1 μL of human serum for 10 min. The diluted primary antibody was used with cells for 30 min at 4° C., and then after washing secondary antibody was added for 30 min at 4C. The cells were rinsed with 3 mL of washing buffer, then stained for 10 min with 7-AAD, and FACS analysis was performed on FACS Calibur (BD Biosciences).

### Cytotoxicity Assay

Real-time impedance-based cytotoxicity assays (RTCA) using CELLigence system (Agilent) were used with Daudi and multiple myeloma cell lines. In brief, 1×10⁴ target cells were seeded into 96-well E-plates covered with CD40 for leukemia cells or CD9 antibodies for multiple myeloma cells to attach cells to the plates (Agilent/Acea Biosciences, San Diego, CA, USA). The next day, the medium was removed and replaced with AIM V-AlbuMAX medium containing 10% FBS±1×10⁵ effector cells at different (Effector to Target cells) E:T ratios in triplicate. The cells were monitored for another 24-48 h with the RTCA system, and impedance was plotted over time. Cytotoxicity percent was calculated as (impedance of target cells without effector cells minus impedance of target cells with effector cells)/impedance of target cells without effector cells×100). For Nalm-6 cells, luciferase-positive, EGFP-positive cells were treated with NK and CD19-CAR-NK cells at different E:T ratios. The cytotoxicity was quantified by luciferase assay with luciferase assay substrate from Steady-Glo Luciferase assay system (Promega). The luciferase-positive alive cells were normalized to untreated Nalm-6-luc+ cells in duplicates, and the percentage of cytotoxicity was calculated for NK and CAR-NK cells at different E:T ratios.

### IFN-Gamma Secretion Assay by ELISA

Nonadherent target cells were cultured with the effector cells at different effector to target (E:T) ratio in U-bottom 96-well plates with 200 μL of AIM V-AlbuMAX medium containing 10% FBS, in triplicate. After 16 h, the top 150 μL of medium was transferred to V-bottom 96-well plates and centrifuged at 300×g for 5 min. The top 120 μL of supernatant was transferred to a new 96-well plate and analyzed by ELISA for human IFN-γ levels using the R&D Systems Human IFN-gamma Quantikine Kit (Minneapolis, MN, USA) according to the manufacturer's protocol. The supernatant after RTCA with adherent target cells was collected and analyzed as above.

### NSG Mouse Model and Imaging

Six-week-old NSG mice (Jackson Laboratories, Bar Harbor, ME, USA) were housed in accordance with the Institutional Animal Care and Use Committee (IACUC) (#LUM-001). Each mouse was injected subcutaneously on day 0 with 100 μL of 1×10⁵ Nalm-6-luciferase positive cells in sterile medium. 5×10⁶ NK or CAR-NK cells in NK medium were injected intravenously on days 1, 3, 6, and 8. Imaging was done after luciferin injection using Xenogen Ivis System (Perkin Elmer, Waltham, MA, USA). Quantification was done by measuring bioluminescence (BLI) in photons/sec signals.

### Statistical Analyses

Comparisons between two groups were performed by Student's t-test. Differences with p<0.05 were considered significant. GraphPad software 9.5 version was used to prepare graph.

### Example 1. Preparation of Linearized DNA Template for In Vitro Transcription

DNA was digested with appropriate restriction Bgl II (AGATCT) or Asc I (GGCGCGCC) enzyme which cut DNA at 3′-end after poly A tail at 37° C. overnight following manufacturer's protocol. The digested DNA was treated with 50-100 μg/mL Proteinase K and 0.5% SDS for 30 minutes at 50° C. Then phenol/chloroform extraction and ethanol precipitation of DNA was performed. The DNA was used for in vitro RNA transcription reaction.

### Example 2. Preparing mRNA by In Vitro Transcription Reaction

For DNA templates with T7AG promoter, we used the below protocol.

### 2.1. The In Vitro Transcription Reaction was Done by Below Protocol:

The DNA template for generating RNA had T7AG promoter in front of coding sequence of protein. The reaction was the following:

Standard RNA Synthesis Protocol using the HiScribe T7 mRNA Kit with CleanCap Reagent AG (NEB #E2080) was used as described below:
1. Set up the following reaction at room temperature in the following order for RNA without pseudo-UTP:

| | 20 μl | | Final conc. or |
|---|---|---|---|
| Components | reaction | | amount |
| Nuclease-free water | X | μl | |
| 10X T7 CleanCap Reagent AG | 2 | μl | |
| Reaction Buffer | | | |
| ATP (60 mM) | 2 | μl | 6 mM final |
| UTP (50 mM) | 2 | μl | 5 mM final |
| CTP (50 mM) | 2 | μl | 5 mM final |
| GTP (50 mM) | 2 | μl | 5 mM final |
| Cap Analog (40 mM) | 2 | μl | 4 mM final |
| Template DNA | X | μl | 1 μg |

| | |
|---|---|
| T7 RNA Polymerase Mix | 2 μl |

2. Gently mix the reaction by pipetting up and down and microfuge briefly. Incubate at 37° C. for 2 hours.
3. Bring the reaction volume up to 50 μl with nuclease-free water. Add 2 μl of DNase I, mix well and incubate at 37° C. for 15 minutes
4. Proceed with mRNA purification
   For Reaction with Pseudo-UTP we Use the Following Protocol:
1. Thaw the necessary components, keep the T7 RNA Polymerase Mix on ice.
2. Mix and pulse-spin in a microfuge to collect the solutions to the bottom of the tubes.
3. Set up the reaction at room temperature in the following order:

| | | final concentration |
|---|---|---|
| | volume | mM |
| Nuclease-free Water | | |
| 10X Reaction Buffer | 2 | 0.5x |
| 100 mM ATP | 2 | 5 mM final |
| 100 mM GTP | 2 | 5 mM final |
| 100 mM methyl-Pseudo-UTP | 2 | 5 mM final |
| (N-1081) | | |
| 100 mM CTP | 2 | 5 mM final |
| 100 mM CleanCapAG (3′ OMe | 1.6 | 4 mM final |
| N-7413) | | |
| Linear Template DNA | | 1 μg total |
| T7 RNA Polymerase Mix | 4 | |
| Total | 40 μl | |

4. Gently mix the reaction by pipetting up and down and microfuge briefly. Incubate at 37° C. for 2 hours.
5. Optional: The reaction volume can be up to 50 μl with nuclease-free water. Add 2 μl of DNase I, mix well and incubate at 37° C. for 15 minutes.

### 2.2. Cleaning In Vitro Transcribed RNA

For cleaning RNA we used NEB The Monarch RNA Cleanup Kit (T2050) according to manufacturer's protocol.

### 2.3. Assessing RNA Yield

The concentration of RNA can be determined by diluting an aliquot of the preparation (usually a 1:50 to 1:100 dilution) in 1×TE (10 mM Tris-HCl pH 8, 1 mM EDTA) buffer, and reading the absorbance in a spectrophotometer at 260 nm. The concentration (μg/mL) of RNA is therefore calculated as follows: A260×dilution factor×40 μg/mL.

2.4. mRNA In Vitro Transcription

mRNA was in vitro transcribed from a DNA template with T7AG promoter using the HiScribe T7 mRNA Kit with CleanCap Reagent AG (NEB #E2080). In vitro transcription detail reaction conditions are shown in Examples 2.1. For GFP coding sequence inserted into DNA template vector for in vitro transcription with T7 AG promoter in front and 5′UTR, 3′UTR flanking open reading frame of the codon sequence and 152 poly A tail after the stop codon. For CD19 CAR, CD19 scFv (FMC63) Flag tag-CD28-CD3 sequence was used for inserting into the above vector (26). For BCMA-CAR, humanized BCMA scFv-41BB-CD3 CAR was used in the DNA template vector (27). In brief, a DNA template, 0.5×T7 CleanCap Reagent AG Reaction Buffer, 5 mM of ATP, CTP, pseudo-UTP, and GTP were added to 4 mM of CleanCapAG and T7 polymerase mix for 2 h at 37° C. After DNAse I treatment for 15 min at 37° C., the mRNA was purified with the Monarch RNA Cleanup Kit (T2050) according to the manufacturer's protocol. After each reaction, mRNA was checked on agarose gel with molecular weight ladder, and concentration of mRNA was detected with Nanodrop.

### Example 3. CAR DNA Template Sequences

### 3.1A. BCMA-CAR DNA Template

We used BCMACAR DNA templates with 5′ and 3′ UTR and poly A tail (PMC1538). T7 promoter underlined; CAR is shown in bold, 150 nucleotide poly A tail is shown in Italics

(SEQ ID NO: 1)
TAATACGACTCACTATAAGGAGAAAGCTTacatttgcttctgacacaactgtgttcactagcaacctcaaacag
acaccATGGCCTTACCAGTGACCGCCTTGCTCCTGCCGCTGGCCTTGCTGCTCCA
CGCCGCCAGGCCGGCTAGCCAGGTGCAGCTGGTGCAGAGCGGCGCGGAAGTG
AAAAAACCGGGCAGCAGCGTGAAAGTGAGCTGCAAAGCGAGCGGCTATACCTT
TACCAGCTATGTGATGCATTGGGTGCGCCAGGCGCCGGGCCAGGGCCTGGAAT
GGATGGGCTATATTATTCCGTATAACGATGCGACCAAATATAACGAAAAATTTA
AAGGCCGCGTGACCATTACCGCGGATAAAAGCACCAGCACCGCGTATATGGAA
CTGAGCAGCCTGCGCAGCGAAGATACCGCGGTGTATTATTGCGCGCGCTATAA
CTATGATGGCTATTTTGATGTGTGGGGCCAGGGCACCCTGGTGACCGTGAGCA
GCGGCGGCGGCGGCAGCGGCGGCGGCGGCAGCGGCGGCGGCGGCAGCGATG
TGGTGATGACCCAGAGCCCGGCGTTTCTGAGCGTGACCCCGGGCGAAAAAGTG
ACCATTACCTGCCGCGCGAGCCAGAGCATTAGCGATTATCTGCATTGGTATCA
GCAGAAACCGGATCAGGCGCCGAAACTGCTGATTAAATATGCGAGCCAGAGCA
TTAGCGGCGTGCCGAGCCGCTTTAGCGGCAGCGGCAGCGGCACCGATTTTACC
TTTACCATTAGCAGCCTGGAAGCGGAAGATGCGGCGACCTATTATTGCCAGAA
CGGCCATAGCTTTCCGCCGACCTTTGGCGGCGGCACCAAAGTGGAAATTAAAC
TCGAGAAGCCCACCACGACGCCAGCGCCGCGACCACCAACACCGGCGCCCACC
ATCGCGTCGCAGCCCCTGTCCCTGCGCCCAGAGGCGAGCCGGCCAGCGGCGG
GGGGCGCAGTGCACACGAGGGGGCTGGACTTCGCCAGTGATAAGCCCTTTTGG
GTGCTGGTGGTGGTTGGTGGAGTCCTGGCTTGCTATAGCTTGCTAGTAACAGT
GGCCTTTATTATTTTCTGGGTGAAACGGGGCAGAAAGAAACTCCTGTATATATT
CAAACAACCATTTATGAGACCAGTACAAACTACTCAAGAGGAAGATGGCTGTA
GCTGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGA
AGGCCTGTACAATGAACTGCAGAAAGATAAGATGGCGGAGGCCTACAGTGAGA
TTGGGATGAAAGGCGAGCGCCGGAGGGGCAAGGGGCACGATGGCCTTTACCA
GGGTCTCAGTACAGCCACCAAGGACACCTACGACGCCCTTCACATGCAGGCCC
TGCCCCCTCGCTAAGctcgctttcttgctgtccaatttctattaaaggttcctttgttccctaagtccaactactaaa
ctgggggatattatgaagggccttgagcatctggattctgcctaataaaaaacatttattttcattgcagctcgcttt
cttgctgtccaatttctattaaaggttcctttgttccctaagtccaactactaaactgggggatattatgaagggcct
tgagcatctggattctgcctaataaaaaacatttattttcattgcaGTCGACTCTAGAAAAAAAAAAAAAAAAAAAAA
AAAAAAAAAAAAAAAAAAAAGGATCCCCGGGCGAGCTCCCAAAAAAAAAAAAAAAAAAAAAAAAAAAAAACCGAATTC
CTGCAGCTCGAGAAAAAAAAAAAAAAAAAAAAAAAAAAAAAAAAAAAAAAAAA

### T7 Promoter

(SEQ ID NO: 2)
TAATACGACTCACTATAAG
5′UTR
(SEQ ID NO: 3)
GAGAAAGCTTacatttgcttctgacacaactgtgttcactagcaacct
caaacagacacc

### BCMA-CAR Nucleotide Sequence:

(SEQ ID NO: 4)
TAATACGACTCACTATAAGGAGAAAGCTTacatttgcttctgacacaactgtgttcactagcaacctcaaacag
acaccATGGCCTTACCAGTGACCGCCTTGCTCCTGCCGCTGGCCTTGCTGCTCCA
CGCCGCCAGGCCGGCTAGCCAGGTGCAGCTGGTGCAGAGCGGCGCGGAAGTG
AAAAAACCGGGCAGCAGCGTGAAAGTGAGCTGCAAAGCGAGCGGCTATACCTT
TACCAGCTATGTGATGCATTGGGTGCGCCAGGCGCCGGGCCAGGGCCTGGAAT
GGATGGGCTATATTATTCCGTATAACGATGCGACCAAATATAACGAAAAATTTA
AAGGCCGCGTGACCATTACCGCGGATAAAAGCACCAGCACCGCGTATATGGAA
CTGAGCAGCCTGCGCAGCGAAGATACCGCGGTGTATTATTGCGCGCGCTATAA
CTATGATGGCTATTTTGATGTGTGGGGCCAGGGCACCCTGGTGACCGTGAGCA
GCGGCGGCGGCGGCAGCGGCGGCGGCGGCAGCGGCGGCGGCGGCAGCGATG
TGGTGATGACCCAGAGCCCGGCGTTTCTGAGCGTGACCCCGGGCGAAAAAGTG
ACCATTACCTGCCGCGCGAGCCAGAGCATTAGCGATTATCTGCATTGGTATCA
GCAGAAACCGGATCAGGCGCCGAAACTGCTGATTAAATATGCGAGCCAGAGCA
TTAGCGGCGTGCCGAGCCGCTTTAGCGGCAGCGGCAGCGGCACCGATTTTACC
TTTACCATTAGCAGCCTGGAAGCGGAAGATGCGGCGACCTATTATTGCCAGAA
CGGCCATAGCTTTCCGCCGACCTTTGGCGGCGGCACCAAAGTGGAAATTAAAC
TCGAGAAGCCCACCACGACGCCAGCGCCGCGACCACCAACACCGGCGCCCACC
ATCGCGTCGCAGCCCCTGTCCCTGCGCCCAGAGGCGAGCCGGCCAGCGGCGG
GGGGCGCAGTGCACACGAGGGGGCTGGACTTCGCCAGTGATAAGCCCTTTTGG
GTGCTGGTGGTGGTTGGTGGAGTCCTGGCTTGCTATAGCTTGCTAGTAACAGT
GGCCTTTATTATTTTCTGGGTGAAACGGGGCAGAAAGAAACTCCTGTATATATT
CAAACAACCATTTATGAGACCAGTACAAACTACTCAAGAGGAAGATGGCTGTA
GCTGCCGATTTCCAGAAGAAGAAGAAGGAGGATGTGAACTGAGAGTGAAGTTC
AGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGA
AGGCCTGTACAATGAACTGCAGAAAGATAAGATGGCGGAGGCCTACAGTGAGA
TTGGGATGAAAGGCGAGCGCCGGAGGGGCAAGGGGCACGATGGCCTTTACCA
GGGTCTCAGTACAGCCACCAAGGACACCTACGACGCCCTTCACATGCAGGCCC
TGCCCCCTCGCTAA
3′UTR
(SEQ ID NO: 5)
Gctcgctttcttgctgtccaatttctattaaaggttcctttgttccctaagtccaactactaaactgggggatatta
tgaagggccttgagcatctggattctgcctaataaaaaacatttattttcattgcagctcgctttcttgctgtccaa
tttctattaaaggttcctttgttccctaagtccaactactaaactgggggatattatgaagggccttgagcatctgg
attctgcctaataaaaaacatttattttcattgcaGTCGACTCTAG
Poly A tail (152 nucleotide),
(SEQ ID NO: 6)
AAAAAAAAAAAAAAAAAAAAAAAAAAAAAAAAAAAAAAAAAGGATCCCCGGGCGAGCTCCC
AAAAAAAAAAAAAAAAAAAAAAAAAAAAAACCGAATTCCTGCAGCTCGAGAAAAAAAAAAAA
AAAAAAAAAAAAAAAAAAAAAAAAAAAAA
Translated BCMA-41BB-CD3 CAR amino acid sequence
(SEQ ID NO: 7)
MALPVTALLLPLALLLHAARPASQVQLVQSGAEVKKPGSSVKVSCKASGYTFTSYVMH
WVRQAPGQGLEWMGYIIPYNDATKYNEKFKGRVTITADKSTSTAYMELSSLRSEDTAV
YYCARYNYDGYFDVWGQGTLVTVSSGGGGSGGGGSGGGGSDVVMTQSPAFLSVTPGE
KVTITCRASQSISDYLHWYQQKPDQAPKLLIKYASQSISGVPSRFSGSGSGTDFTFTISSLE
AEDAATYYCONGHSFPPTFGGGTKVEIKLEKPTTTPAPRPPTPAPTIASQPLSLRPEASRP
AAGGAVHTRGLDFASDKPFWVLVVVGGVLACYSLLVTVAFIIFWVKRGRKKLLYIFKQ
PFMRPVQTTQEEDGCSCRFPEEEEGGCELRVKFSRSADAPAYQQGQNQLYNELNLGRRE
EYDVLDKRRGRDPEMGGKPQRRKNPQEGLYNELQKDKMAEAYSEIGMKGERRRGKG
HDGLYQGLSTATKDTYDALHMQALPPR

### 3.1B. BCMA-CAR DNA Template (Uridine Depleted RNA)

We also used DNA template (capital letters below) with T substituted to C or A nucleotide bases in the third of three-nucleotide codon (underlined below, in bold) without changing amino acid to generate uridine depleted RNA sequence of BCMA (PMC1767) for higher expression of CAR.

The nucleic acid sequence of PMC1767 is shown below. The amino acid sequence is the same as shown in 3.1A above.

(SEQ ID NO: 8)
TAATACGACTCACTATAAGGAGAAAGCTTacatttgcttctgacacaactgtgttcactagcaacctcaaacag
acacc
ATG GCG CTC CCA GTG ACT GCC CTT CTG TTG CCA TTG GCC TTG CTT TTG CAC
GCC GCG AGG CCC GCC TCC CAA GTG CAA CTC GTT CAG TCC GGG GCG GAG GT
C AAA AAG CCA GGC TCC TCC GTC AAG GTA TCC TGT AAG GCG TCC GGA TAC A
CA TTC ACC TCC TAC GTC ATG CAC TGG GTC AGA CAA GCA CCG GGC CAA GGG
CTC GAG TGG ATG GGC TAC ATT ATA CCG TAC AAC GAC GCG ACG AAG TAC AA
C GAA AAG TTC AAA GGG AGA GTA ACC ATA ACG GCC GAC AAA AGC ACA AGC
ACT GCG TAC ATG GAA CTC TCC TCC CTC CGA TCC GAA GAT ACC GCA GTA TAC
TAC TGT GCC AGA TAC AAC TAC GAT GGT TAC TTC GAC GTC TGG GGA CAG GG
C ACC CTG GTA ACA GTA TCA TCA GGA GGA GGG GGC AGC GGA GGC GGC GGA
TCA GGG GGC GGC GGC AGC GAC GTC GTG ATG ACG CAG AGC CCG GCA TTC CT
C TCT GTC ACA CCC GGA GAG AAG GTC ACA ATC ACT TGC CGC GCA TCC CAA A
GC ATA TCA GAC TAC CTG CAC TGG TAC CAG CAG AAA CCC GAC CAA GCC CCC
AAA CTC TTG ATA AAG TAC GCC AGT CAA AGC ATA TCA GGA GTC CCC TCC CG
G TTC AGT GGC AGT GGC TCC GGA ACG GAC TTC ACG TTC ACC ATC TCA TCA TT
G GAA GCG GAA GAC GCG GCA ACA TAC TAC TGC CAA AAT GGC CAC AGC TTC C
CG CCC ACG TTC GGG GGC GGA ACA AAA GTC GAA ATA AAG TTG GAG AAA CCC
ACC ACT ACA CCA GCC CCC AGA CCA CCC ACT CCC GCA CCG ACC ATC GCG AG
C CAG CCA CTC TCT CTG AGA CCC GAG GCC TCA CGC CCG GCC GCA GGG GGC G
CG GTC CAC ACG CGC GGG CTC GAT TTT GCC TCC GAC AAA CCC TTC TGG GTC
CTG GTC GTA GTA GGA GGA GTC CTG GCC TGC TAC TCC TTG TTG GTA ACC GTT
GCG TTC ATC ATC TTC TGG GTC AAG AGA GGC CGA AAG AAA CTG CTC TAC AT
C TTC AAG CAA CCC TTC ATG CGC CCG GTC CAA ACA ACA CAA GAA GAG GAC G
GC TGC TCA TGC CGC TTT CCG GAG GAG GAG GAA GGG GGC TGT GAA TTG AGG
GTG AAA TTC AGC CGG TCT GCG GAC GCC CCC GCC TAC CAA CAG GGC CAG AA
T CAA CTC TAC AAC GAA CTC AAC TTG GGG AGA CGC GAG GAA TAC GAT GTA C
TG GAT AAG CGA CGC GGG CGC GAC CCT GAG ATG GGG GGC AAG CCC CAG AG
G AGG AAG AAC CCC CAA GAG GGC CTG TAC AAC GAG CTG CAG AAG GAC AAA
ATG GCG GAG GCC TAC TCA GAG ATC GGG ATG AAG GGC GAA CGG AGA CGC G
GA AAA GGG CAC GAC GGG CTC TAC CAA GGC TTG TCA ACA GCT ACC AAG GAC
ACC TAT GAC GCG CTC CAC ATG CAA GCG TTG CCA CCC AGA TAAgctcgctttcttgctgt
ccaatttctattaaaggttcctttgttccctaagtccaactactaaactgggggatattatgaagggccttgagcatctggat
tctgcctaataaaaaacatttattttcattgcagctcgctttcttgctgtccaatttctattaaaggttcctttgttccctaa
gtccaactactaaactgggggatattatgaagggccttgagcatctggattctgcctaataaaaaacatttattttcattgca
GTCGACTCTAGAAAAAAAAAAAAAAAAAAAAAAAAAAAAAAAAAAAAAAAAAGGATCCCCGGGCGAGCTCCCAAAAAAAAA
AAAAAAAAAAAAAAAAAAAAACCGAATTCCTGCAGCTCGAGAAAAAAAAAAAAAA
AAAAAAAAAAAAAAAAAAAAAAAAAAAGATCTGGCGCGCCGTAATCATGTCATAG
CTGTTTCCTGTGTGAAATTGTTATCCGCTCACAATTCCACACAACATACGAGCCGGA
AGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTC
AGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCACGC
TCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGCTACAGGC
ATCGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGA
GTACTCAACCAAGTCATTCTGAGAATAGTGTATGCGGCGACCGAGTTGCTCTTGCCC
GGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACC
ATTATTATCATGACATTAACCTATAAAAATAGGCGTATCACGAGGCCCTTTCGTCTC
GCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAG
CGGGTGTTGGCGGGTGTCGGGGCTGGCTTAACTATGCGGCATCAGAGCAGATTGTAC
TGAGAGTGCACCATTCGACGCTCTCCCTTATGCGACTCCTGCATTAGGAAGCAGCCC
AGTAGTAGGTTGAGGCCGTTGAGCACCGCCGCCGCAAGGAATGGTGCATGCAAGGA
GATGGCGCCCAACAGTCCCCCGGCCACGGGGCCTGCCACCATACCCACGCCGAAAC
AAGCGCTCATGAGCCCGAAGTGGCGAGCCCGATCTTCCCCATCGGTGATGTCGGCG
ATATAGGCGCCAGCAACCGCACCTGTGGCGCCGGTGATGCCGGCCACGATGCGTCC
GGCGTAGAGGATCTGGCTAGCGATGACCCTGCTGATTGGTTCGCTGACCATTTCCGG
GTGCGGGACGGCGTTACCAGAAACTCAGAAGGTTCGTCCAACCAAACCGACTCTGA
CGGCAGTTTACGAGAGAGATGATAGGGTCTGCTTCAGTAAGCCAGATGCTACACAA
TTAGGCTTGTACATATTGTCGTTAGAACGCGGCTACAATTAATACATAACCTTATGT
ATCATACACATACG

The vector we used was with Kanamycin R gene instead of Amp.

### 3.2A. CD19-CAR DNA Template

We also used CD19-CAR (PMC1643) to generate CD19-41BB-CD3 CAR-RNA. CAR DNA template is with 5′ and 3′ UTR and poly A tail. T7 promoter underlined; CD19-CAR is shown in bold, 150 nucleotide poly A tail is shown in Italics

(SEQ ID NO: 9)
TAATACGACTCACTATAAGGAGAAAGCTTacatttgcttctgacacaactgtgttcactagcaacctcaaacag
acacc
ATGGCCTTACCAGTGACCGCCTTGCTCCTGCCGCTGGCCTTGCTGCTCCACGC
CGCCAGGCCGGACATCCAGATGACACAGACTACATCCTCCCTGTCTGCCTCTCT
GGGAGACAGAGTCACCATCAGTTGCAGGGCAAGTCAGGACATTAGTAAATATT
TAAATTGGTATCAGCAGAAACCAGATGGAACTGTTAAACTCCTGATCTACCATA
CATCAAGATTACACTCAGGAGTCCCATCAAGGTTCAGTGGCAGTGGGTCTGGA
ACAGATTATTCTCTCACCATTAGCAACCTGGAGCAAGAAGATATTGCCACTTAC
TTTTGCCAACAGGGTAATACGCTTCCGTACACGTTCGGAGGGGGGACTAAGTT
GGAAATAACAGGTGGCGGTGGCAGCGGCGGTGGTGGTTCCGGAGGCGGCGGT
TCTGAGGTGAAACTGCAGGAGTCAGGACCTGGCCTGGTGGCGCCCTCACAGAG
CCTGTCCGTCACATGCACTGTCTCAGGGGTCTCATTACCCGACTATGGTGTAAG
CTGGATTCGCCAGCCTCCACGAAAGGGTCTGGAGTGGCTGGGAGTAATATGGG
GTAGTGAAACCACATACTATAATTCAGCTCTCAAATCCAGACTGACCATCATCA
AGGACAACTCCAAGAGCCAAGTTTTCTTAAAAATGAACAGTCTGCAAACTGATG
ACACAGCCATTTACTACTGTGCCAAACATTATTACTACGGTGGTAGCTATGCTA
TGGACTACTGGGGTCAAGGAACCTCAGTCACCGTCTCCTCAACCACGACGCCA
GCGCCGCGACCACCAACACCGGCGCCCACCATCGCGTCGCAGCCCCTGTCCCT
GCGCCCAGAGGCGTGCCGGCCAGCGGCGGGGGGCGCAGTGCACACGAGGGGG
CTGGACTTCGCCTGTGATATCTACATCTGGGCGCCCCTGGCCGGGACTTGTGG
GGTCCTTCTCCTGTCACTGGTTATCACCCTTTACTGCAAACGGGGCAGAAAGAA
ACTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAAC
TGAGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATGTTT
TGGACAAGAGACGTGGCCGGGACCCTGAGATGGGGGGAAAGCCGAGAAGGAA
GAACCCTCAGGAAGGCCTGTACAATGAACTGCAGAAAGATAAGATGGCGGAGG
CCTACAGTGAGATTGGGATGAAAGGCGAGCGCCGGAGGGGCAAGGGGCACGA
TGGCCTTTACCAGGGTCTCAGTACAGCCACCAAGGACACCTACGACGCCCTTC
ACATGCAGGCCCTGCCCCCTCGCTGATAG
TAAGctcgctttcttgctgtccaatttctattaaaggttcctttgttccctaagtccaactactaaactgggggatattatgaa
gggccttgagcatctggattctgcctaataaaaaacatttattttcattgcagctcgctttcttgctgtccaatttctattaaa
ggttcctttgttccctaagtccaactactaaactgggggatattatgaagggccttgagcatctggattctgcctaataaaaaa
catttattttcattgcaGTCGACTCTAGAAAAAAAAAAAAAAAAAAAAAAAAAAAAAAAAAAAAAAAAAGGATCCCCGGGCGAG
CTCCCAAAAAAAAAAAAAAAAAAAAAAAAAAAAAACCGAATTCCTGCAGCTCGAGA
AAAAAAAAAAAAAAAAAAAAAAAAAAAAAAAAAAAAAAAA
Amino acid sequence of CD19-41BB-CD3 CAR
(SEQ ID NO: 10)
MALPVTALLLPLALLLHAARPDIQMTQTTSSLSASLGDRVTISCRASQDISKYLNWYQQ
KPDGTVKLLIYHTSRLHSGVPSRFSGSGSGTDYSLTISNLEQEDIATYFCQQGNTLPYTFG
GGTKLEITGGGGSGGGGSGGGGSEVKLQESGPGLVAPSQSLSVTCTVSGVSLPDYGVSW
IRQPPRKGLEWLGVIWGSETTYYNSALKSRLTIIKDNSKSQVFLKMNSLQTDDTAIYYCA
KHYYYGGSYAMDYWGQGTSVTVSSTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEED
GCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKD
TYDALHMQALPPR

### 3.2B. CD19-CAR DNA Template (CD28 Signal and Tag)

We also used CD19-CAR with CD28 signaling domain and Flag tag or TF tag after CD19 scFv.

PMC1637 with CD19Flag-CD28-CD3 template for mRNA is shown below. T7AG promoter bold underlined, CD19-Flag tag-Cd28-CD3 sequence is shown in bold. Flag tag is in bold, italics, underlined.

(SEQ ID NO: 11)
TAATACGACTCACTATAAGGAGAAAGCTTacatttgcttctgacacaactgtgttcactagcaacctcaaacag
acacc
ATGCTTCTCCTGGTGACAAGCCTTCTGCTCTGTGAGTTACCACACCCAGCATTC
CTCCTGATCCCAGACATCCAGATGACACAGACTACATCCTCCCTGTCTGCCTCT
CTGGGAGACAGAGTCACCATCAGTTGCAGGGCAAGTCAGGACATTAGTAAATA
TTTAAATTGGTATCAGCAGAAACCAGATGGAACTGTTAAACTCCTGATCTACCA
TACATCAAGATTACACTCAGGAGTCCCATCAAGGTTCAGTGGCAGTGGGTCTG
GAACAGATTATTCTCTCACCATTAGCAACCTGGAGCAAGAAGATATTGCCACTT
ACTTTTGCCAACAGGGTAATACGCTTCCGTACACGTTCGGAGGGGGGACTAAG
TTGGAAATAACAGGCTCCACCTCTGGATCCGGCAAGCCCGGATCTGGCGAGGG
ATCCACCAAGGGCGAGGTGAAACTGCAGGAGTCAGGACCTGGCCTGGTGGCG
CCCTCACAGAGCCTGTCCGTCACATGCACTGTCTCAGGGGTCTCATTACCCGAC
TATGGTGTAAGCTGGATTCGCCAGCCTCCACGAAAGGGTCTGGAGTGGCTGGG
AGTAATATGGGGTAGTGAAACCACATACTATAATTCAGCTCTCAAATCCAGACT
GACCATCATCAAGGACAACTCCAAGAGCCAAGTTTTCTTAAAAATGAACAGTCT
GCAAACTGATGACACAGCCATTTACTACTGTGCCAAACATTATTACTACGGTGG
TAGCTATGCTATGGACTACTGGGGTCAAGGAACCTCAGTCACCGTCTCCTCAG
CGGCCGCAGACTACAAAGACGATGACGACAAGATTGAAGTTATGTATCCTCCTCC
TTACCTAGACAATGAGAAGAGCAATGGAACCATTATCCATGTGAAAGGGAAAC
ACCTTTGTCCAAGTCCCCTATTTCCCGGACCTTCTAAGCCCTTTTGGGTGCTGG
TGGTGGTTGGGGGAGTCCTGGCTTGCTATAGCTTGCTAGTAACAGTGGCCTTT
ATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATG
CCCCACCACGCGACTTCGCAGCCTATCGCTCCAGAGTGAAGTTCAGCAGGAGC
GCAGACGCCCCCGCGTACCAGCAGGGCCAGAACCAGCTCTATAACGAGCTCAA
TCTAGGACGAAGAGAGGAGTACGATGTTTTGGACAAGAGACGTGGCCGGGACC
CTGAGATGGGGGGAAAGCCGAGAAGGAAGAACCCTCAGGAAGGCCTGTACAA
TGAACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGTAC
AGCCACCAAGGACACCTACGACGCCCTTCACATGCAGGCCCTGCCCCCTCGCT
AAGctcgctttcttgctgtccaatttctattaaaggttcctttgttccctaagtccaactactaaactgggggatattatgaagggc
cttgagcatctggattctgcctaataaaaaacatttattttcattgcagctcgctttcttgctgtccaatttctattaaaggttcct
ttgttccctaagtccaactactaaactgggggatattatgaagggccttgagcatctggattctgcctaataaaaaacatttatttt
cattgcaGTCGACTCTAGAAAAAAAAAAAAAAAAAAAAAAAAAAAAAAAAAAAAAAAAAGGATCCCCGGGCGAGCT
CCCAAAAAAAAAAAAAAAAAAAAAAAAAAAAAACCGAATTCCTGCAGCTCGAGAA
AAAAAAAAAAAAAAAAAAAAAAAAAAAAAAAAAAAAAAAGATCTGGCGCGCCGT
AATCATGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACT
CACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCT
CTTCCGCTTCCTCGCTCACTGACTCGCTGCGCTCGGTCGTTCGGCTGCGGCGAGCGG
TATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGAC
GCTCAAGTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACA
CGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAG
CAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATC
TAAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTGT
AGATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGA
GTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAA
AGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACG
TCTAAGAAACCATTATTATCATGACATTAACCTATAAAAATAGGCGTATCACGAGGC
CCTTTCGTCTCGCGCGTTTCGGTGATGACGGTGAAAACCTCTGACACATGCAGCTCC
CGGAGACGGTCACAGCTTGTCTGTAAGCGGATGCCGGGAGCAGACAAGCCCGTCAG
GGCGCGTCAGCGGGTGTTGGCGGGTGTCGGGGCTGGCTTAACTATGCGGCATCAGA
GCAGATTGTACTGAGAGTGCACCATTCGACGCTCTCCCTTATGCGACTCCTGCATTA
GGAAGCAGCCCAGTAGTAGGTTGAGGCCGTTGAGCACCGCCGCCGCAAGGAATGGT
GCATGCAAGGAGATGGCGCCCAACAGTCCCCCGGCCACGGGGCCTGCCACCATACC
CACGCCGAAACAAGCGCTCATGAGCCCGAAGTGGCGAGCCCGATCTTCCCCATCGG
TGATGTCGGCGATATAGGCGCCAGCAACCGCACCTGTGGCGCCGGTGATGCCGGCC
ACGATGCGTCCGGCGTAGAGGATCTGGCTAGCGATGACCCTGCTGATTGGTTCGCTG
ACCATTTCCGGGTGCGGGACGGCGTTACCAGAAACTCAGAAGGTTCGTCCAACCAA
ACCGACTCTGACGGCAGTTTACGAGAGAGATGATAGGGTCTGCTTCAGTAAGCCAG
ATGCTACACAATTAGGCTTGTACATATTGTCGTTAGAACGCGGCTACAATTAATACA
TAACCTTATGTATCATACACATACG

Amino acid sequence: Flag tag underlined.

(SEQ ID NO: 12)
MLLLVTSLLLCELPHPAFLLIPDIQMTQTTSSLSASLGDRVTISCRASQDISKYLNWYQQK
PDGTVKLLIYHTSRLHSGVPSRFSGSGSGTDYSLTISNLEQEDIATYFCQQGNTLPYTFGG
GTKLEITGSTSGSGKPGSGEGSTKGEVKLQESGPGLVAPSQSLSVTCTVSGVSLPDYGVS
WIRQPPRKGLEWLGVIWGSETTYYNSALKSRLTIIKDNSKSQVFLKMNSLQTDDTAIYY
CAKHYYYGGSYAMDYWGQGTSVTVSSAAADYKDDDDKIEVMYPPPYLDNEKSNGTII
HVKGKHLCPSPLFPGPSKPFWVLVVVGGVLACYSLLVTVAFIIFWVRSKRSRLLHSDYM
NMTPRRPGPTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGERRRGKGH
DGLYQGLSTATKDTYDALHMQALPPR

TF tag instead of Flag tag was used in PMC2039. T7Ag promoter underlined, coding sequence CD19TF-28-CD is in bold.

(SEQ ID NO: 13)
TAATACGACTCACTATAAGGAGAAAGCTTacatttgcttctgacacaactgtgttcactagcaacctcaaacag
acaccATGCTTCTCCTGGTGACAAGCCTTCTGCTCTGTGAGTTACCACACCCAGCA
TTCCTCCTGATCCCAGACATCCAGATGACACAGACTACATCCTCCCTGTCTGCC
TCTCTGGGAGACAGAGTCACCATCAGTTGCAGGGCAAGTCAGGACATTAGTAA
ATATTTAAATTGGTATCAGCAGAAACCAGATGGAACTGTTAAACTCCTGATCTA
CCATACATCAAGATTACACTCAGGAGTCCCATCAAGGTTCAGTGGCAGTGGGT
CTGGAACAGATTATTCTCTCACCATTAGCAACCTGGAGCAAGAAGATATTGCCA
CTTACTTTTGCCAACAGGGTAATACGCTTCCGTACACGTTCGGAGGGGGGACT
AAGTTGGAAATAACAGGCTCCACCTCTGGATCCGGCAAGCCCGGATCTGGCGA
GGGATCCACCAAGGGCGAGGTGAAACTGCAGGAGTCAGGACCTGGCCTGGTG
GCGCCCTCACAGAGCCTGTCCGTCACATGCACTGTCTCAGGGGTCTCATTACC
CGACTATGGTGTAAGCTGGATTCGCCAGCCTCCACGAAAGGGTCTGGAGTGGC
TGGGAGTAATATGGGGTAGTGAAACCACATACTATAATTCAGCTCTCAAATCCA
GACTGACCATCATCAAGGACAACTCCAAGAGCCAAGTTTTCTTAAAAATGAACA
GTCTGCAAACTGATGACACAGCCATTTACTACTGTGCCAAACATTATTACTACG
GTGGTAGCTATGCTATGGACTACTGGGGTCAAGGAACCTCAGTCACCGTCTCC
TCAGCGGCCGCAaaaaacccggatccgtgggcgaaaaacctgaacgaaaaagattatATTGAAGTTATG
TATCCTCCTCCTTACCTAGACAATGAGAAGAGCAATGGAACCATTATCCATGTG
AAAGGGAAACACCTTTGTCCAAGTCCCCTATTTCCCGGACCTTCTAAGCCCTTT
TGGGTGCTGGTGGTGGTTGGGGGAGTCCTGGCTTGCTATAGCTTGCTAGTAAC
AGTGGCCTTTATTATTTTCTGGGTGAGGAGTAAGAGGAGCAGGCTCCTGCACA
GTGACTACATGAACATGACTCCCCGCCGCCCCGGGCCCACCCGCAAGCATTAC
CAGCCCTATGCCCCACCACGCGACTTCGCAGCCTATCGCTCCAGAGTGAAGTT
CAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGT
GGCCGGGACCCTGAGATGGGGGGAAAGCCGAGAAGGAAGAACCCTCAGGAAG
GCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGG
GTCTCAGTACAGCCACCAAGGACACCTACGACGCCCTTCACATGCAGGCCCTG
CCCCCTCGCTAAGctcgctttcttgctgtccaatttctattaaaggttcctttgttccctaagtccaactactaaactg
ggggatattatgaagggccttgagcatctggattctgcctaataaaaaacatttattttcattgcagctcgctttcttg
ctgtccaatttctattaaaggttcctttgttccctaagtccaactactaaactgggggatattatgaagggccttgagc
atctggattctgcctaataaaaaacatttattttcattgcaGTCGACTCTAGAAAAAAAAAAAAAAAAAAAAAAAAAAA
AAAAAAAAAAAAAAGGATCCCCGGGCGAGCTCCCAAAAAAAAAAAAAAAAAAAAAAAAAAAAAACCGAATTCCTG
CAGCTCGAGAAAAAAAAAAAAAAAAAAAAAAAAAAAAAAAAAAAAAAAAAGATC
TGGCGCGCCGTAATCATGTCATAGCTGTTTCCTGTGTGAAATTGTTATCCGCTCACAA
TTCCACACAACATACGAGCCGGAAGCATAAAGTGTAAAGCCTGGGGTGCCTAATGA
GTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCG
TATTGGGCGCTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGG
GGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGTGCACGAAC
CCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTT
TAAATCAATCTAAAGTATATATGAGTAAACTTGGTCTGACAGTTACCAATGCTTAAT
CAGTGAGGCACCTATCTCAGCGATCTGTCTATTTCGTTCATCCATAGTTGCCTGACTC
CCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCA
ACGTTGTTGCCATTGCTACAGGCATCGTGGTGTCACGCTCGTCGTTTGGTATGGCTTC
ATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGT
GTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAG
CAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATC
TTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACTCTTCC
TTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAG
TGCCACCTGACGTCTAAGAAACCATTATTATCATGACATTAACCTATAAAAATAGGC
GTATCACGAGGCCCTTTCGTCTCGCGCGTTTCGGTGATGACGGTGAAAACCTCTGAC
ACATGCAGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCTTAACTA
TGCGGCATCAGAGCAGATTGTACTGAGAGTGCACCATTCGACGCTCTCCCTTATGCG
ACTCCTGCATTAGGAAGCAGCCCAGTAGTAGGTTGAGGCCGTTGAGCACCGCCGCC
GCAAGGAATGGTGCATGCAAGGAGATGGCGCCCAACAGTCCCCCGGCCACGGGGCC
TGCCACCATACCCACGCCGAAACAAGCGCTCATGAGCCCGAAGTGGCGAGCCCGAT
CTTCCCCATCGGTGATGTCGGCGATATAGGCGCCAGCAACCGCACCTGTGGCGCCGG
TGATGCCGGCCACGATGCGTCCGGCGTAGAGGATCTGGCTAGCGATGACCCTGCTG
ATTGGTTCGCTGACCATTTCCGGGTGCGGGACGGCGTTACCAGAAACTCAGAAGGTT
CGTCCAACCAAACCGACTCTGACGGCAGTTTACGAGAGAGATGATAGGGTCTGCTTC
AGTAAGCCAGATGCTACACAATTAGGCTTGTACATATTGTCGTTAGAACGCGGCTAC
AATTAATACATAACCTTATGTATCATACACATACG

The amino acid is shown below:

(SEQ ID NO: 14)
MLLLVTSLLLCELPHPAFLLIPDIQMTQTTSSLSASLGDRVTISCRASQDISKYLNWYQQK
PDGTVKLLIYHTSRLHSGVPSRFSGSGSGTDYSLTISNLEQEDIATYFCQQGNTLPYTFGG
GTKLEITGSTSGSGKPGSGEGSTKGEVKLQESGPGLVAPSQSLSVTCTVSGVSLPDYGVS
WIRQPPRKGLEWLGVIWGSETTYYNSALKSRLTIIKDNSKSQVFLKMNSLQTDDTAIYY
CAKHYYYGGSYAMDYWGQGTSVTVSSAAAKNPDPWAKNLNEKDYIEVMYPPPYLDN
EKSNGTIIHVKGKHLCPSPLFPGPSKPFWVLVVVGGVLACYSLLVTVAFIIFWVRSKRSR
LLHSDYMNMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYSEIGMKGE
RRRGKGHDGLYQGLSTATKDTYDALHMQALPPR

### 3.3. GFP DNA Template

We also used GFP in DNA template with different 5′UTR and 3′UTR and poly A tail.

DNA template for preparing PMC1634 (GFP RNA):

(SEQ ID NO: 15)
TAATACGACTCACTATAAGGAGAGGCCAGGAACCGTAAAAAGGCCGCGTTGCTGG
CGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTGAT
CCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACG
CTCAGTGGAACGAAAACTCACGTTAAGGGATTTTGGTCATGAGAAGCTTGAGAATA
AACTAGTATTCTTCTGGTCCCCACAGACTCAGAGAGAACCCGCCaccATGGTGAGCA
AGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCCCGT
GCCCTGGCCCACCCTCGTGACCACCCTGACCTACGGCGTGCAGTGCTTCAGCC
GCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGA
AGGGCATCGACTTCAAGGAGGACGGCAACATCCTGGGGCACAAGCTGGAGTAC
AACTACAACAGCCACAACGTCTATATCATGGCCGACAAGCAGAAGAACGGCAT
CAAGGTGAACTTCAAGATCCGCCACAACATCGAGGACGGCAGCGTGCAGCTCG
CCGACCACTACCAGCAGAACACCCCCATCGGCGACGGCCCCGTGCTGCTGCCC
GACAACCACTACCTGAGCACCCAGTCCGCCCTGAGCAAAGACCCCAACGAGAA
GCGCGATCACATGGTCCTGCTGGAGTTCGTGACCGCCGCCGGGATCACTCTCG
GCATGGACGAGCTGTACAAGtagTGATAAgaCTCGAGCTGGTACTGCATGCACGCA
ATGCTAGCTGCCCCTTTCCCGTCCTGGGTACCCCGAGTCTCCCCCGACCTCGGGTCCC
AGGTATGCTCCCACCTCCACCTGCCCCACTCACCACCTCTGCTAGTTCCAGACACCT
CCCAAGCACGCAGCAATGCAGCTCAAAACGCTTAGCCTAGCCACACCCCCACGGGA
AACAGCAGTGATTAACCTTTAGCAATAAACGAAAGTTTAACTAAGCTATACTAACCC
CAGGGTTGGTCAATTTCGTGCCAGCCACACCCTGGAGCTAGCGTCGACTCTAGAAAA
AAAAAAAAAAAAAAAAAAAAAAAAAAAAAAAAAAAAAGGATCCCCGGGCGAGCT
CCCAAAAAAAAAAAAAAAAAAAAAAAAAAAAAACCGAATTCCTGCAGCTCGAGAA
AAAAAAAAAAAAAAAAAAAAAAAAAAAAAAAAAAAAAAA

### T7 Promoter

(SEQ ID NO: 2)
TAATACGACTCACTATAAG_

### 5′UTR:

(SEQ ID NO: 16)
GAGAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACT
GGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAA
GCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACT
CACGTTAAGGGATTTTGGTCATGAGAAGCTTGAGAATAAACTAGTATTCTTCTGGTC
CCCACAGACTCAGAGAGAACCCGCCacc
GFP
(SEQ ID NO: 17)
ATGGTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCGAG
GGCGATGCCACCTACGGCAAGCTGACCCTGAAGTTCATCTGCACCACCGGCAA
GCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCC
ATGCCCGAAGGCTACGTCCAGGAGCGCACCATCTTCTTCAAGGACGACGGCAA
CTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAG
CTGGAGTACAACTACAACAGCCACAACGTCTATATCATGGCCGACAAGCAGAA
GAACGGCATCAAGGTGAACTTCAAGATCCGCCACAACATCGAGGACGGCAGCG
TGCAGCTCGCCGACCACTACCAGCAGAACACCCCCATCGGCGACGGCCCCGTG
CTGCTGCCCGACAACCACTACCTGAGCACCCAGTCCGCCCTGAGCAAAGACCC
CAACGAGAAGCGCGATCACATGGTCCTGCTGGAGTTCGTGACCGCCGCCGGGA
TCACTCTCGGCATGGACGAGCTGTACAAGtagTGATAA

### Translate Amino Acid GFP:

(SEQ ID NO: 18)
MVSKGEELFTGVVPILVELDGDVNGHKFSVSGEGEGDATYGKLTLKFIC
TTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLL
PDNHYLSTQSALSKDPNEKRDHMVLLEFVTAAGITLGMDELYK

### 3′UTR

(SEQ ID NO: 19)
gaCTCGAGCTGGTACTGCATGCACGCAATGCTAGCTGCCCCTTTCCCGT
CCTGGGTACCCCGAGTCTCCCCCGACCTCGGGTCCCAGGTATGCTCCCA
CCTCCACCTGCCCCACTCACCACCTCTGCTAGTTCCAGACACCTCCCAA
GCACGCAGCAATGCAGCTCAAAACGCTTAGCCTAGCCACACCCCCACGG
GAAACAGCAGTGATTAACCTTTAGCAATAAACGAAAGTTTAACTAAGCT
ATACTAACCCCAGGGTTGGTCAATTTCGTGCCAGCCACACCCTGGAGCT
AGCGTCGACTCTAG

### Poly a Tail (152 Nucleotide)

(SEQ ID NO: 6)
AAAAAAAAAAAAAAAAAAAAAAAAAAAAAAAAAAAAAAAAAGGATCCCC
GGGCGAGCTCCCAAAAAAAAAAAAAAAAAAAAAAAAAAAAAACCGAATT
CCTGCAGCTCGAGAAAAAAAAAAAAAAAAAAAAAAAAAAAAAAAAAAAA
AAAAA

### Example 4. NK Cell Isolation and Expansion

NK cells were isolated from PBMC using NK Cell Isolation Kit, Human (Miltenyi Biotec) according to the manufacturer's protocol. The NK cells were expanded using K562-41-BBL, IL-21 feeder cells pre-treated with Mitomycin C (Sigma) using gas-permeable static cell culture flasks (G-Rex) (Wilson-Wolf) (25). The medium for expansion was AIM-V, 10% FBS with IL-2 [10 ng/mL] and IL-15 [5 ng/mL]. NK cells were frozen using NutriFreez D10 Cryopreservation Medium, without phenol red (Satorius).

### Example 5. Effective Expansion of NK Cells and Transfection with GFP-mRNA-LNP

K562-4-1BB-IL21 K562 cell line was used for expansion of NK cells (see Example 5). FACS show expression of 4-1BBL and IL21 in K562 after transduction with lentivirus encoding 4-1BB and IL21.

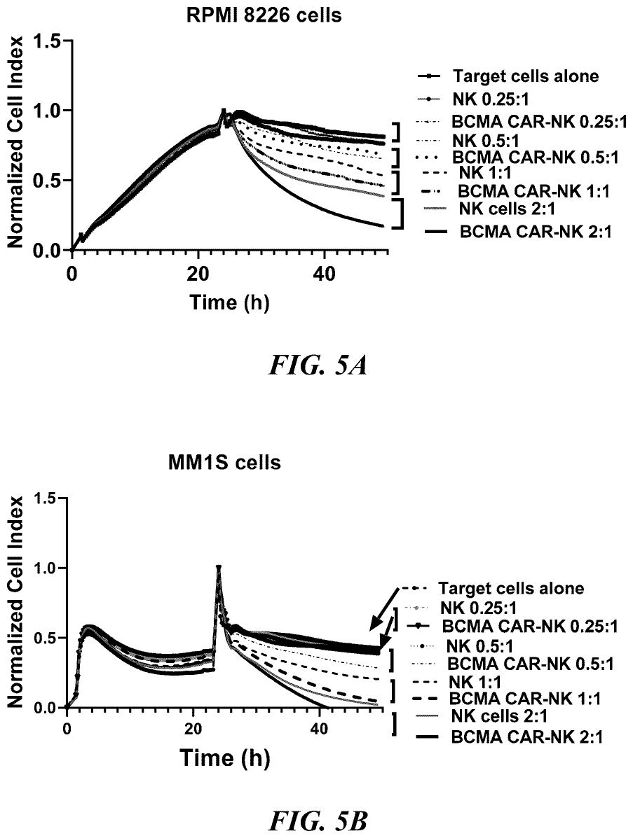

FIG. 3 shows the expansion of NK cells with K562-4-BB, IL-21 K562 cell line. FIG. 3 shows an average of fold of expansion of NK cell expansion from two different donors is shown. The NK cells expanded with these feeder cells up to 5754-fold in 18 days using G-Rex system.

In characterization of expanded NK cells, FACS with anti-CD56 and CD3 antibody showed 98% CD56-positive, CD3-negative expanded NK cells.

To check transfection efficiency of NK cells with mRNA-LNP, we prepared GFP mRNA embedded into LNP using NanoSystem and transfected expanded NK cells. Expanded NK cells were transfected with GFP mRNA-LNP, frozen in D10 cryopreservation medium and then thawed and cultivated in NK expansion medium at different time points to check for stability of GFP expression.

The transfection efficiency was high as 98% NK cells were GFP-positive 16 hours after transfection and maintained this efficiency after freezing in D10 cryopreservation medium and thawing up to 72 hours. The NK cells were 94% positive 72 hours after freezing/thawing in culture. Thus, NK cells were efficiently expanded with K562-41BB ligand, IL21-positive cells and effectively transfected with GFP mRNA-LNP.

### Example 6. mRNA-LNP Generation and Transfection of NK Cells

### Materials

SM-102:8-[(2-hydroxyethyl) [6-oxo-6-(undecyloxy)hexyl]amino]-octanoic acid, 1-octylnonyl ester; CAS number: 2089251-47-6
DMG-PEG2000: 1,2-dimyristoyl-rac-glycero-3-methoxypolyethylene glycol-2000
DSPC: Distearoylphosphatidylcholine
Cholesterol (Sigma)

To generate an mRNA-LNP complex, an aqueous solution of mRNA in 100 mM sodium acetate (pH 4.0) was combined with a lipid mix containing the ethanol phase of SM-102 (Cayman), DSPC (Avanti), cholesterol (Sigma), and DMG-PEG2000 (Cayman) (at a molar % ratio of 50:10:38.5:1.5, respectively).

To generate mRNA-LNP the above mix was processed with PreciGenome Flex S System (San Jose, CA, USA) at a flow rate ratio of 3:1 (aqueous:organic phase). The mRNA-LNPs were purified and concentrated using Amicon® Ultra-15 centrifugal filter units (30-100 kDa). The polydispersity index (PDI), size, and zeta-potential of mRNA-LNPs were detected using an Anton Paar Litesizer 500 System.

The size of nanoparticles is confirmed using Dynamic Light Scattering (DLS) system. The size of RNA-LNP nanoparticles is usually in the range of 90-105 nM or 75-100 nM.

LNP can also be prepared with other ionizing lipids such as 50 mM 1 ml (23.15 mM LNP-0315; 4.7 mM DSPC; 21.35 mM Cholesterol; 0.8 mM ALC-0159) which is formulated at ratio 46.3:9.4:42.7:1.6 mol %, respectively.

### Example 7. BCMA CAR RNA-LNP Transfected to NK Cells Generates BCMA-CAR-NK Cells

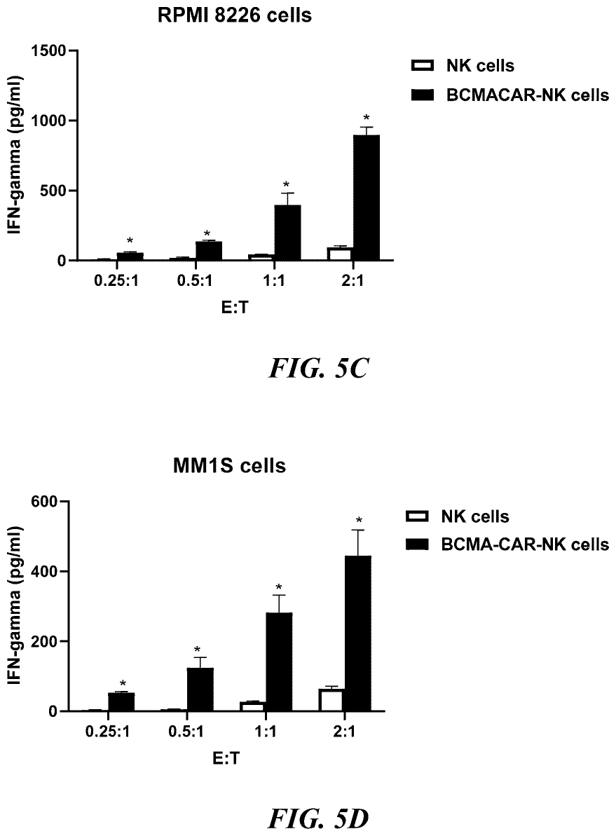

BCMA CAR RNA was generated from template with (PMC1538) DNA template. RNA was embedded to LNP and was transfected to expanded NK cells. Expression of CAR was detected 24, 48, 72 and 96-144 hours after transfection of RNA-LNP. Similar results were obtained with PMC1767 (uridine-depleted template). We found that NK cells should be expanded in the range of 500-1000 to get high expression of CAR. We found that different donors varied in CAR expression and that the donor selection was important for preparation of NK cells due to variability of CAR-positive cells. We generated 648-fold expanded NK cells with >95% of BCMA+ CAR+ NK cells (see FIG. 4).

### Example 8. BCMA-CAR-NK Cells (Frozen/Thawed) Generated with BCMA-CAR-RNA-LNP had Cytotoxic Activity Against BCMA-Positive Cells

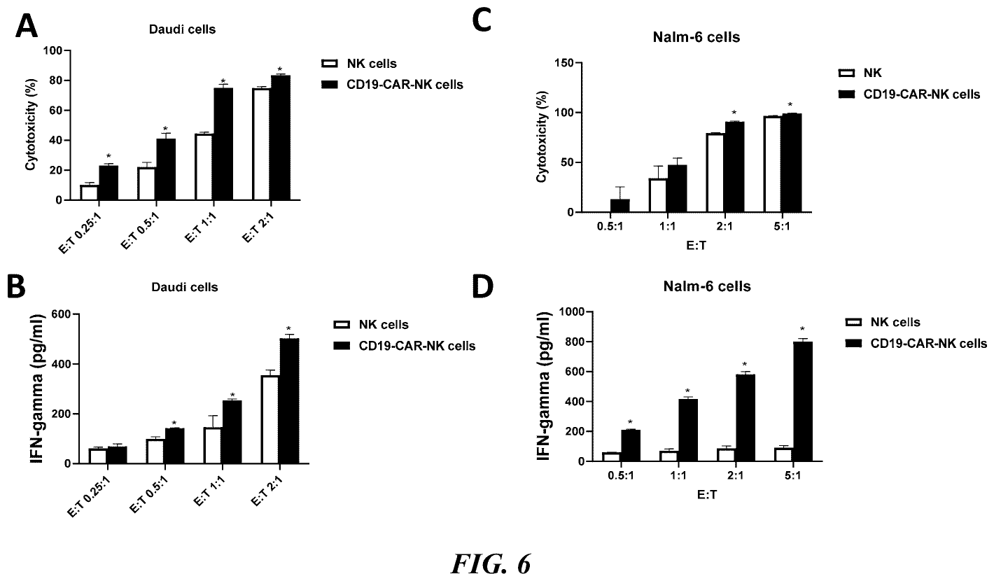

To test functional activity of CAR-NK, first we used BCMA-CAR-NK in Real-time Cytotoxicity assay with multiple myeloma RPMI8226 cells at different E:T (effector to target cell) ratios (FIG. 5A). BCMA-CAR-NK killed RPMI-8226 cells in a dose dependent manner more than NK cells (FIG. 5A). The same result was obtained with another multiple myeloma cell line, MM1S (FIG. 5B). BCMA-CAR-NK cells killed target cells more than NK cells (FIG. 5B).

The supernatant was collected after killing assay for testing secretion of IFN-gamma by NK and CAR-NK cells (FIGS. 5C, 5D). BCMA-CAR-NK cells secreted IFN-gamma in a dose-dependent manner significantly higher level than NK cells with target RPMI-8226 cells (FIG. 5C) and with MM1S target cells (FIG. 5D). Thus, transfection of BCMA CAR mRNA-LNP into NK cells generates functional BCMA CAR-NK cells with high killing activity and secretion of IFN-gamma.

### Example 9. CD19-CAR-NK Cells Kill Leukemia Cells and Secrete Higher Level of IFN-Gamma than NK Cells

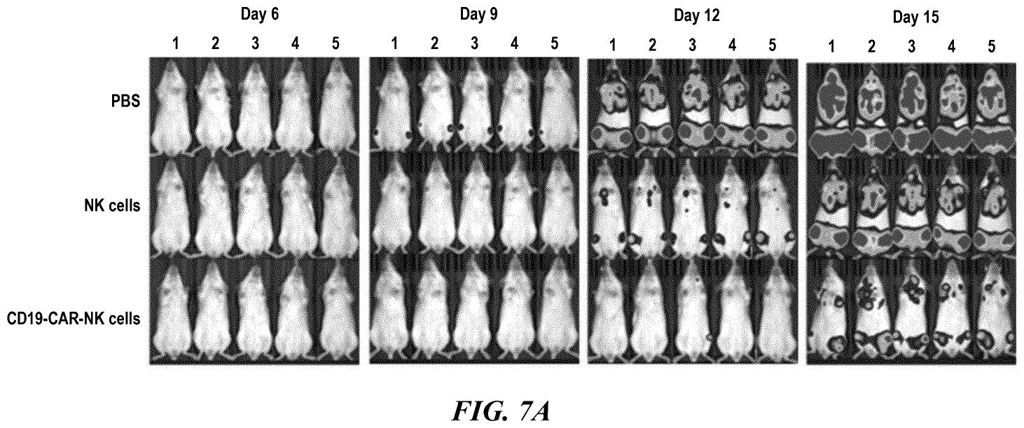

CD19-CAR-NK cells generated by transfection of CD19-CAR-mRNA-LNP was tested functionally using killing and IFN-gamma secretion ELISA assays. CD19-CAR-NK killed Daudi target cells significantly more than NK cells using Real-time cytotoxicity assay (FIG. 6A). CD19-CAR-NK secreted IFN-gamma significantly higher level than NK cells (FIG. 6B). The same result was obtained in luciferase killing assay with Nalm-6-luciferase positive target cells (FIG. 6C). CD19-CAR-NK killed Nalm-6-luciferase-positive cells significantly more than NK cells (FIG. 6C). CD19 CAR-NK secreted IFN-gamma in a dose-dependent manner at significantly higher level than NK cells (FIG. 6D). Thus, transfection of CD19-CAR mRNA-LNP into NK cells generates highly functional CD19-CAR-NK cells against leukemia tumor cells.

### Example 10. In Vivo Activity of CD19-CAR-NK Cells

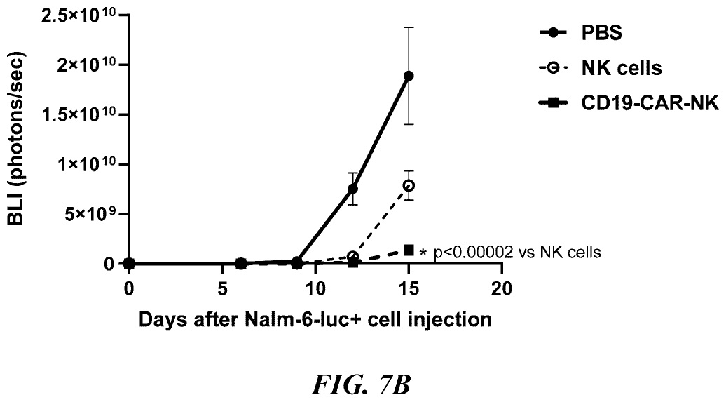

To test in vivo activity of CAR-NK, we used Nalm-6-luciferase cells in NSG mouse model. First, 1×10⁵ Nalm-6-luc+ cells were injected intravenously into mice. Then, 5×10⁶ frozen NK and CD19 CAR-NK cells were injected into mice at days 1,3,6 and 8. Both NK and CD19-CAR-NK blocked Nalm-6 tumor growth (FIG. 7A). Importantly, CD19-CAR-NK blocked Nalm-6-luc+ tumor growth significantly more than NK cells (p<0.00002) at day 15 (FIG. 7B). This, CD19-CAR-NK cells generated with mRNA-LNP expressed high efficacy against Nalm-6 lymphoma tumors.

### Example 11. Freezing Medium for CAR-NK to Maintain High Viability and CAR Expression

For developing allogenic CAR-NK, it is important to have a freezing medium to keep high percent viable cells and high CAR expression. CryoStor® CS5 is a uniquely formulated serum-free, animal component-free and defined cryopreservation medium containing 5% dimethyl sulfoxide (DMSO), which is designed to preserve cells in low temperature environments (−80° C. to −196° C.). D10 medium (NutriFreez D10 Cryopreservation Medium) is a ready-to-use solution for the animal component-free, xeno-free, serum-free cryopreservation of human embryonic stem (ES), induced pluripotent stem (iPS) and mesenchymal stem cells. We tried 4 different mediums to freeze CAR-NK cells and found that CS5 and D10 were optimal for preserving CAR expression and providing high viability of CAR-NK cells. In both medium, CAR-NK were frozen and thawed and maintained high viability and high expression after 24-48 hours of thawing.

GFP (PMC1634) RNA-LNP were transfected to NK cells, frozen in D10 medium and kept in NK medium for different time points after thawing. They were tested for percent of GFP-positive cells and intensity of count after f thawing. GFP was expressed in 95.6% of cells at 48 hours, in 94.5% of cells at 72 hours, after thawing.

mRNA-LNP complex transfected BCMA-CAR-NK cells were frozen in D10 medium and they were detected by FACS with anti-mouse FAB antibody against BCMA post-thawing for different period of time at 0, 24, and 48, hours after thawing, 99.8%, 98.6%, and 71,2%, positive BCMA-CAR-NK cells were detected, respectively.

### REFERENCES

1. R. Abrantes, H. O. Duarte, C. Gomes, S. Walchli and C. A. Reis: CAR-Ts: new perspectives in cancer therapy. FEBS Lett, 596 (4), 403-416 (2022) doi: 10.1002/1873-3468.14270
2. R. C. Sterner and R. M. Sterner: CAR-T cell therapy: current limitations and potential strategies. Blood Cancer J, 11 (4), 69 (2021) doi: 10.1038/s41408-021-00459-7
3. M. V. Maus and C. H. June: CARTs on the road for myeloma. Clin Cancer Res, 20 (15), 3899-901 (2014) doi: 10.1158/1078-0432.CCR-14-0721
4. M. Ruella and C. H. June: Chimeric Antigen Receptor T cells for B Cell Neoplasms: Choose the Right CAR for You. Curr Hematol Malig Rep, 11 (5), 368-84 (2016) doi: 10.1007/s11899-016-0336-z
5. M. Daher, L. Melo Garcia, Y. Li and K. Rezvani: CAR-NK cells: the next wave of cellular therapy for cancer. Clin Transl Immunology, 10 (4), e1274 (2021) doi: 10.1002/cti2.1274
6. S. S. Neelapu, S. Tummala, P. Kebriaei, W. Wierda, C. Gutierrez, F. L. Locke, K. V. Komanduri, Y. Lin, N. Jain, N. Daver, J. Westin, A. M. Gulbis, M. E. Loghin, J. F. de Groot, S. Adkins, S. E. Davis, K. Rezvani, P. Hwu and E. J. Shpall: Chimeric antigen receptor T-cell therapy-assessment and management of toxicities. Nat Rev Clin Oncol, 15 (1), 47-62 (2018) doi: 10.1038/nrclinonc.2017.148
7. E. Liu, Y. Tong, G. Dotti, H. Shaim, B. Savoldo, M. Mukherjee, J. Orange, X. Wan, X. Lu, A. Reynolds, M. Gagea, P. Banerjee, R. Cai, M. H. Bdaiwi, R. Basar, M. Muftuoglu, L. Li, D. Marin, W. Wierda, M. Keating, R. Champlin, E. Shpall and K. Rezvani: Cord blood NK cells engineered to express IL-15 and a CD19-targeted CAR show long-term persistence and potent anti-tumor activity. Leukemia (2017) doi: 10.1038/leu.2017.226
8. S. Z. F. Kiaei, A. Nouralishahi, M. Ghasemirad, M. Barkhordar, S. Ghaffari, H. Kheradjoo, M. Saleh, S. Mohammadzadehsaliani and Z. Molaeipour: Advances in natural killer cell therapies for breast cancer. Immunol Cell Biol (2023) doi: 10.1111/imcb.12658
9. M. K. Kilgour, D. J. Bastin, S. H. Lee, M. Ardolino, S. McComb and A. Visram: Advancements in CAR-NK therapy: lessons to be learned from CAR-T therapy. Front Immunol, 14, 1166038 (2023) doi: 10.3389/fimmu.2023.1166038
10. A. Merino, J. Maakaron and V. Bachanova: Advances in NK cell therapy for hematologic malignancies: NK source, persistence and tumor targeting. Blood Rev, 60, 101073 (2023) doi: 10.1016/j.blre.2023.101073
11. E. Liu, D. Marin, P. Banerjee, H. A. Macapinlac, P. Thompson, R. Basar, L. Nassif Kerbauy, B. Overman, P. Thall, M. Kaplan, V. Nandivada, I. Kaur, A. Nunez Cortes, K. Cao, M. Daher, C. Hosing, E. N. Cohen, P. Kebriaei, R. Mehta, S. Neelapu, Y. Nieto, M. Wang, W. Wierda, M. Keating, R. Champlin, E. J. Shpall and K. Rezvani: Use of CAR-Transduced Natural Killer Cells in CD19-Positive Lymphoid Tumors. N Engl J Med, 382 (6), 545-553 (2020) doi: 10.1056/NEJMoa1910607
12. L. Herrera, S. Santos, M. A. Vesga, J. Anguita, I. Martin-Ruiz, T. Carrascosa, M. Juan and C. Eguizabal: Adult peripheral blood and umbilical cord blood NK cells are good sources for effective CAR therapy against CD19 positive leukemic cells. Sci Rep, 9 (1), 18729 (2019) doi: 10.1038/s41598-019-55239-y
13. A. Leivas, A. Valeri, L. Cordoba, A. Garcia-Ortiz, A. Ortiz, L. Sanchez-Vega, O. Grana-Castro, L. Fernandez, G. Carreno-Tarragona, M. Perez, D. Megias, M. L. Paciello, J. Sanchez-Pina, A. Perez-Martinez, D. A. Lee, D. J. Powell, Jr., P. Rio and J. Martinez-Lopez: NKG2D-CAR-transduced natural killer cells efficiently target multiple myeloma. Blood Cancer J, 11 (8), 146 (2021) doi: 10.1038/s41408-021-00537-w
14. J. Chu, Y. Deng, D. M. Benson, S. He, T. Hughes, J. Zhang, Y. Peng, H. Mao, L. Yi, K. Ghoshal, X. He, S. M. Devine, X. Zhang, M. A. Caligiuri, C. C. Hofmeister and J. Yu: CS1-specific chimeric antigen receptor (CAR)-engineered natural killer cells enhance in vitro and in vivo antitumor activity against human multiple myeloma. Leukemia, 28 (4), 917-27 (2014) doi: 10.1038/leu.2013.279
15. J. Han, J. Chu, W. Keung Chan, J. Zhang, Y. Wang, J. B. Cohen, A. Victor, W. H. Meisen, S. H. Kim, P. Grandi, Q. E. Wang, X. He, I. Nakano, E. A. Chiocca, J. C. Glorioso Iii, B. Kaur, M. A. Caligiuri and J. Yu: CAR-Engineered NK Cells Targeting Wild-Type EGFR and EGFRvIII Enhance Killing of Glioblastoma and Patient-Derived Glioblastoma Stem Cells. Sci Rep, 5, 11483 (2015) doi: 10.1038/srep11483
16. J. Han, J. Chu, W. Keung Chan, J. Zhang, Y. Wang, J. B. Cohen, A. Victor, W. H. Meisen, S. H. Kim, P. Grandi, Q. E. Wang, X. He, I. Nakano, E. A. Chiocca, J. C. Glorioso, 3rd, B. Kaur, M. A. Caligiuri and J. Yu: CAR-Engineered NK Cells Targeting Wild-Type EGFR and EGFRvIII Enhance Killing of Glioblastoma and Patient-Derived Glioblastoma Stem Cells. Sci Rep, 5, 11483 (2015) doi: 10.1038/srep11483
17. H. Liu, B. Yang, T. Sun, L. Lin, Y. Hu, M. Deng, J. Yang, T. Liu, J. Li, S. Sun and S. Jiao: Specific growth inhibition of ErbB2-expressing human breast cancer cells by genetically modified NK-92 cells. Oncol Rep, 33 (1), 95-102 (2015) doi: 10.3892/or.2014.3548
18. K. Schonfeld, C. Sahm, C. Zhang, S. Naundorf, C. Brendel, M. Odendahl, P. Nowakowska, H. Bonig, U. Kohl, S. Kloess, S. Kohler, H. Holtgreve-Grez, A. Jauch, M. Schmidt, R. Schubert, K. Kuhlcke, E. Seifried, H. G. Klingemann, M. A. Rieger, T. Tonn, M. Grez and W. S. Wels: Selective inhibition of tumor growth by clonal NK cells expressing an ErbB2/HER2-specific chimeric antigen receptor. Mol Ther, 23 (2), 330-8 (2015) doi: 10.1038/mt.2014.219
19. Y. Li, D. L. Hermanson, B. S. Moriarity and D. S. Kaufman: Human iPSC-Derived Natural Killer Cells Engineered with Chimeric Antigen Receptors Enhance Anti-tumor Activity. Cell Stem Cell, 23 (2), 181-192 e5 (2018) doi: 10.1016/j.stem.2018.06.002
20. T. Bexte, L. M. Reindl and E. Ullrich: Non-viral technologies can pave the way for CAR-NK cell therapy. J Leukoc Biol (2023) doi: 10.1093/jleuko/qiad074 ADDIN EN.REFLIST
21. V. Golubovskaya, H. Zhou, F. Li, M. Valentine, J. Sun, R. Berahovich, S. Xu, M. Quintanilla, M. C. Ma, J. Sienkiewicz, Y. Huang and L. Wu: Novel CD37, Humanized CD37 and Bi-Specific Humanized CD37-CD19 CAR-T Cells Specifically Target Lymphoma. Cancers (Basel), 13 (5) (2021) doi: 10.3390/cancers13050981

What is claimed is:

1. Natural killer (NK) cells transfected with mRNA and lipid nanoparticles (LNPs) complex, wherein the mRNA comprises (i) 5′-UTR (untranslated region) coding sequence, (ii) a chimeric antigen receptor fusion protein (CAR) coding sequence that target a tumor antigen, (iii) a 3′-UTR coding sequence, and (iv) a poly A tail sequence.

2. The NK cells of claim 1, wherein the tumor antigen is BCMA, Her-2, HER-2-t2A-GM-CSF, CD47, CD19, CS1, or Claudin 18.2.

3. The NK cells of claim 1, wherein the LNPs have an average size of 30-250 nm.

4. The NK cells of claim 1, wherein the LNP comprises (a) 8-[(2-hydroxyethyl) [6-oxo-6-(undecyloxy)hexyl]amino]-octanoic acid, 1-octylnonyl ester (SM-102), distearoylphosphatidylcholine (DSPC), Cholesterol, and 1,2-dimyristoyl-rac-glycero-3-methoxypolyethylene glycol-2000 (DMG-PEG2000), or (b) SM-102, DSPC, Cholesterol, or 1,2-distearoyl-sn-glycero-3-phosphoethanolamine-N-[maleimide (polyethylene glycol)-2000] (DSPE-PEG2000-MAL), or (c) 2-hexyl-decanoic acid, 1,1′-[(4-hydroxybutyl)imino]di-6,1-hexanediyl] ester (ALC-0315), DSPC, Cholesterol, and α-[2-(ditetradecylamino)-2-oxoethyl]-ω-methoxy-poly(oxy-1,2-ethanediyl) (ALC-0159).

5. The NK cells of claim 1, wherein the CAR comprises from N-terminus to C-terminus: (i) a single-chain variable fragment (scFv) against the tumor antigen, (ii) a transmembrane domain, (iii) at least one co-stimulatory domains, and (iv) an activating domain.

6. The NK cells of claim 1, wherein the mRNA further comprises 5′-cap.

7. A method for preparing NK cells of claim 1, comprising the steps of:
obtaining the mRNA-LNP complex;
obtaining NK cells that have been expanded at least 500 fold,
transfecting the mRNA-encapsulated LNPs into the expanded NK cells, and
translating the mRNA in the NK cells to produce CAR.

8. The method of claim 7, wherein the NK cells have been frozen and thawed before transfected with mRNA.

9. The method of claim 7, wherein the mRNA-encapsulated LNPs is transfected into the expanded NK cells in a G-rex (Gas Permeable Rapid expansion) system.

10. A method for treating cancer in a patient, comprising the step of:
administering the NK cells of claim 1 to a patient,
whereby the NK cells target the tumor antigen and kill tumors.
